(19) United States
(12) Patent Application Publication
(10) Pub. No.: US 2024/0279226 A1
(43) Pub. Date: Aug. 22, 2024

(54) ERGOLINE-DERIVED AGONISTS OF THE 5-HT2A RECEPTOR

(71) Applicant: Diamond Therapeutics Inc., Toronto (CA)

(72) Inventor: Methvin ISAAC, Brampton (CA)

(21) Appl. No.: 18/607,966

(22) Filed: Mar. 18, 2024

Related U.S. Application Data
(63) Continuation of application No. PCT/IB2022/000629, filed on Oct. 25, 2022.
(60) Provisional application No. 63/272,082, filed on Oct. 26, 2021.

(51) Int. Cl. C07D 471/06; A61K 31/4745

(52) U.S. Cl. CPC C07D 471/06; A61K 31/4745

(57) ABSTRACT

Provided herein are novel lisuride compounds, processes for their preparation, compositions comprising said compounds, and use in therapy. More particularly, the present disclosure relates to fluorinated and/or deuterated analog useful in the treatment of diseases, disorders or conditions treatable by modulating ther 5-HT2 receptor subtypes.

### CROSS-REFERENCE

This application claims the benefit of U.S. Provisional Patent Application No. 63/272,082 filed on Oct. 26, 2021, which is incorporated herein by reference in its entirety.

### BACKGROUND

Provided herein are novel lisuride compounds, processes for their preparation, compositions comprising said compounds, and use in therapy. More particularly, the present disclosure relates to fluorinated and/or deuterated analog useful in the treatment of diseases, disorders, or conditions treatable by modulating ther 5-HT2 receptor subtypes.

### BRIEF SUMMARY OF THE INVENTION

Provided herein are ergoline-derived 5-HT₂ₐ receptor agonists compounds, pharmaceutical compositions comprising said compounds, and methods for using said compounds for the treatment of diseases.

One embodiment provides a compound, or pharmaceutically acceptable salt or solvate thereof, having the structure of Formula (I):

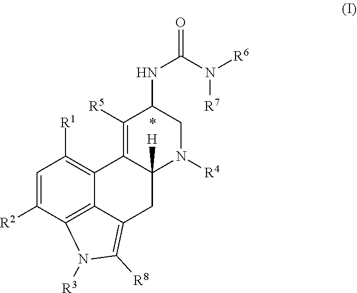

In some embodiments R¹ is H, halogen, alkoxy, haloalkoxy (e.g., OCHF₂, OCF₃), or haloalkyl (e.g., CF₃). In some embodiments, R¹ is H. In some embodiments, R¹ is halogen. In some embodiments, R¹ is alkoxy. In some embodiments, R¹ is haloalkoxy. In some embodiments, R¹ is OCHF₂. In some embodiments, R¹ is OCF₃. In some embodiments, R¹ is haloalkyl. In some embodiments, R¹ is CF₃. In some embodiments, R² is H, halogen, alkoxy, haloalkoxy (e.g., OCHF₂, OCF₃), or haloalkyl (e.g., CF₃). In some embodiments, R² is H. In some embodiments, R² is halogen. In some embodiments, R² is alkoxy. In some embodiments, R² is haloalkoxy. In some embodiments, R² is OCHF₂. In some embodiments, R² is OCF₃. In some embodiments, R² is haloalkyl. In some embodiments, R² is CF₃. In some embodiments, R³ is H, alkyl, or deuteroalkyl. In some embodiments, R³ is H. In some embodiments, R³ is alkyl. In some embodiments, R³ is deuteroalkyl. In some embodiments, R⁴ is alkyl or deteroalkyl. In some embodiments, R⁴ is alkyl. In some embodiments, R⁴ is deuteroalkyl. In some embodiments, R⁵ is H or halogen. In some embodiments, R⁵ is H. In some embodiments, R⁵ is halogen. In some embodiments, R⁶ is optionally substituted C₁₋₆ alkyl or optionally substituted C₁₋₆ alkoxy. In some embodiments, R⁶ is optionally substituted C₁₋₆ alkyl. In some embodiments, R⁶ is C₁₋₆ alkoxy. In some embodiments R⁷ is optionally substituted C₁₋₆ alkyl or optionally substituted C₁₋₆ alkoxy. In some embodiments, R⁷ is C₁₋₆ alkyl. In some embodiments, R⁷ is C₁₋₆ alkoxy. In some embodiments, R⁸ is H or D. In some embodiments, R⁸ is H. In some embodiments, R⁸ is D. In some embodiments, * indicates R or S stereochemistry. In some embodiments, * indicates R stereochemistry. In some embodiments, * indicates S stereochemistry.

In specific embodiments, (a) R⁴ is deuteroalkyl, or (b) R⁶ is optionally substituted C₁₋₆ alkoxy. In specific embodiments, (a) R⁴ is deuteroalkyl, or (b) R⁶ is OCH₃. In specific embodiments, (a) R⁴ is deuteroalkyl, or (b) R⁶ is deuteroalkyl.

One embodiment provides a compound, or pharmaceutically acceptable salt or solvate thereof, having the structure of Formula (Ia):

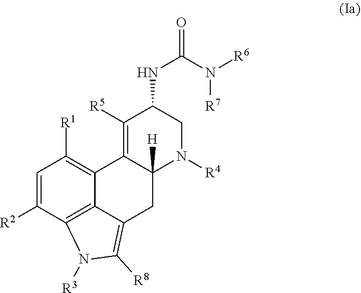

wherein,
R¹ is selected from H, halogen, OMe, CF₃, OCHF₂, and OCF₃;
R² is selected from H, halogen, OMe, CF₃, OCHF₂, and OCF₃;
R³ is selected from H, CH₃ and CD₃;
R⁴ is selected from CH₃ and CD₃;
R⁵ is selected from H or F;
R⁶ is selected from optionally substituted C₁₋₆alkyl, or optionally substituted OC₁-6alkyl;
R⁷ is selected from optionally substituted C₁₋₆alkyl, or optionally substituted OC₁-6alkyl;
R⁸ is selected from H or D;
provided that R¹, R², R³, R⁵, and R⁸ are not H; R⁴ is not CH₃, and R⁶ and R⁷ are not CH₂CH₃.

In some embodiments, R¹ is selected from H, halogen, OMe, CF₃, OCHF₂, and OCF₃. In some embodiments, R¹ is H, halogen OMe, CF₃, OCHF₂, or OCF₃. In some embodiments, R¹ is H. In some embodiments, R¹ is halogen. In some embodiments, R¹ is OMe. In some embodiments, R¹ is CF₃. In some embodiments, R¹ is OCHF₂. In some embodiments, R¹ is OCF₃. In some embodiments, R², is selected from H, halogen, OMe, CF₃, OCHF₂, and OCF₃. In some embodiments, R² is H, halogen, OMe, CF₃, OCHF₂, or OCF₃. In some embodiments, R² is H. In some embodiments, R² is halogen. In some embodiments, R² is OMe. In some embodiments, R² is CF₃. In some embodiments, R² is OCHF₂. In some embodiments, R² is OCF₃. In some embodiments, R³ is selected from H, CH₃ and CD₃. In some embodiments, R³ is H, CH₃ or CD₃. In some embodiments, R³ is H. In some embodiments, R³ is CH₃. In some embodiments, R³ is CD₃. In some embodiments, R⁴ is selected from CH₃ and CD₃. In some embodiments, R⁴ is CH₃ or CD₃. In some embodiments, R⁴ is CH₃. In some embodiments, R⁴ is CD₃. In some embodiments, R⁵ is selected from H or F. In some embodiments, R⁵ is H or F. In some embodiments, R⁵ is H. In some embodiments, R⁵ is F. In some embodiments, R⁶ is selected from optionally substituted C₁₋₆alkyl, or optionally substituted OC₁₋₆alkyl. In some embodiments, R⁶ is optionally substituted C₁₋₆alkyl, or optionally substituted OC₁₋₆alkyl. In some embodiments, R⁶ is optionally substituted C₁₋₆alkyl. In some embodiments, R⁶ is OC₁-6alkyl. In some embodiments, R⁷ is selected from optionally substituted C₁₋₆alkyl, or optionally substituted OC₁₋₆alkyl. In some embodiments, R⁷ is optionally substituted C₁₋₆alkyl, or optionally substituted OC₁₋₆alkyl. In some embodiments, R⁸ is selected from H or D. In some embodiments, R⁸ is H or D. In some embodiments, R⁸ is H. In some embodiments, R⁸ is D. In specific embodiments, R¹, R², R³, R⁵, and R⁸ are not H; R⁴ is not CH₃, and R⁶ and R⁷ are not CH₂CH₃. In some embodiments, the claimed language: provided that R¹, R², R³, R⁵, and R⁸ are not H; R⁴ is not CH₃, and R⁶ and R⁷ are not CH₂CH₃, means: R¹, R², R³, R⁵, and R⁸ are not concurrently H while R⁴ is CH₃ and R⁶ and R⁷ are CH₂CH₃.

One embodiment provides a compound, or pharmaceutically acceptable salt or solvate thereof, having the structure of Formula (II):

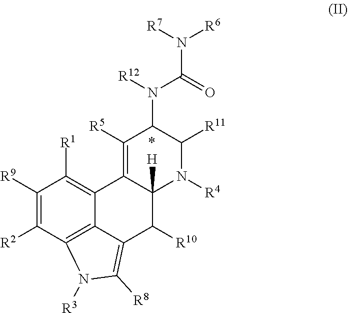

In some embodiments, R¹ is H, halogen, alkoxy, haloalkoxy (e.g., OCHF₂, OCF₃), or haloalkyl (e.g., CF₃). In some embodiments, R¹ is H. In some embodiments, R¹ is halogen. In some embodiments, R¹ is alkoxy. In some embodiments, R¹ is haloalkoxy. In some embodiments, R¹ is haloalkyl. In some embodiments, R¹ is OCHF₂. In some embodiments, R¹ is OCF₃. In some embodiments, R¹ is CF₃. In some embodiments, R² is H, halogen, alkoxy, haloalkoxy (e.g., OCHF₂, OCF₃), or haloalkyl (e.g., CF₃). In some embodiments, R² is H. In some embodiments, R² is halogen. In some embodiments, R² is alkoxy. In some embodiments, R² is haloalkoxy. In some embodiments, R² is OCHF₂. In some embodiments, R² is OCF₃. In some embodiments, R² is haloalkyl. In some embodiments, R² is CF₃. In some embodiments, R³ is H, alkyl, or deteuroalkyl. In some embodiments, R³ is H. In some embodiments, R³ is alkyl. In some embodiments, R³ is alkyl. In some embodiments, R³ is deuteroalkyl. In some embodiments, R⁴ is alkyl or deuteroalkyl. In some embodiments, R⁴ is alkyl. In some embodiments, R⁴ is deuteroalkyl. In some embodiments, R⁵ is H or halogen. In some embodiments, R⁵ is H. In some embodiments, R⁵ is halogen. In some embodiments, R⁶ is optionally substituted C₁₋₆ alkyl or optionally substituted C₁₋₆ alkoxy. In some embodiments, R⁶ is optionally substituted C₁₋₆ alkyl. In some embodiments, R⁶ is optionally substituted C₁₋₆ alkoxy. In some embodiments, R⁷ is optionally substituted C₁₋₆ alkyl or optionally substituted C₁₋₆ alkoxy. In some embodiments, R⁷ is optionally substituted C₁₋₆ alkyl. In some embodiments, R⁷ is optionally substituted C₁₋₆ alkoxy. In some embodiments, R⁸ is H or D. In some embodiments, R⁸ is H. In some embodiments, R⁸ is D. In some embodiments, R⁹ is H, halogen, alkoxy, haloalkoxy (e.g., OCHF₂, OCF₃), or haloalkyl (e.g., CF₃). In some embodiments, R⁹ is H. In some embodiments, R⁹ is halogen. In some embodiments, R⁹ is alkoxy. In some embodiments, R⁹ is haloalkoxy. In some embodiments, R⁹ is OCHF₂. In some embodiments, R⁹ is OCF₃. In some embodiments, R⁹ is haloalkyl. In some embodiments, R⁹ is haloalkyl. In some embodiments, R⁹ is CF₃. In some embodiments, R¹⁰ is H, D, alkyl, cycloalkyl, or deuteroalkyl. In some embodiments, R¹⁰ is H. In some embodiments, R¹⁰ is D. In some embodiments, R¹⁰ is alkyl. In some embodiments, R¹⁰ is cycloalkyl. In some embodiments, R¹⁰ is deuteroalkyl. In some embodiments, R¹¹ is H, D, alkyl, cycloalkyl, or deuteroalkyl. In some embodiments, R¹¹ is H. In some embodiments, R¹¹ is D. In some embodiments, R¹¹ is alkyl. In some embodiments, R¹¹ is cycloalkyl. In some embodiments, R¹¹ is deuteroalkyl. In some embodiments, R¹² is H, alkyl, cycloalkyl, or deuteroalkyl. In some embodiments, R¹² is H. In some embodiments, R¹² is alkyl. In some embodiments, R¹² is cycloalkyl. In some embodiments, R¹² is deuteroalkyl. In some embodiments, * indicates R or S steroechemistry. In some embodiments, * indicates R steroeochemistry. In some embodiments, * indicates S stereochemistry.

In specific embodiments, (a) R⁴ is deuteroalkyl, or (b) R⁶ is optionally substituted C₁₋₆ alkoxy. In specific embodiments, (a) R⁴ is deuteroalkyl, or (b) R⁶ is OCH₃. In specific embodiments, (a) R⁴ is deuteroalkyl, or (b) R⁶ is deuteroalkyl.

One embodiment provides a pharmaceutical composition comprising a compound of Formula (I), or pharmaceutically acceptable salt or solvate thereof, and at least one pharmaceutical excipient. One embodiment provides a pharmaceutical composition comprising a compound of Formula (Ia), or pharmaceutically acceptable salt or solvate thereof, and at least one pharmaceutically acceptable excipient. One embodiment provides a pharmaceutical composition comprising a compound of Formula (II), or pharmaceutically acceptable salt or solvate thereof, and at least one pharmaceutically acceptable excipient.

### INCORPORATION BY REFERENCE

All publications, patents, and patent applications mentioned in this specification are herein incorporated by reference for the specific purposes identified herein.

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a}(where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}—, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a}(where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}—, —SW, —OC(O)—R^{a}, —N(W)₂, —C(O)W, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}—, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises two carbon atoms (e.g., C₂ alkenylene). In other embodiments, an alkenylene comprises five to eight carbon atoms (e.g., C₅-C₈ alkenylene). In other embodiments, an alkenylene comprises three to five carbon atoms (e.g., C₃-C₅ alkenylene). Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkynylene). In other embodiments, an alkynylene comprises five to eight carbon atoms (e.g., C₅-C₈ alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}—, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OW, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{c}-aryl, where R^{c} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O—R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” or “cycloalkyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbomyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical is optionally substituted as defined above.

“Carbocyclylalkynyl” refers to a radical of the formula —R-carbocyclyl where R^{c} is an alkynylene chain as defined above. The alkynylene chain and the carbocyclyl radical is optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical is optionally substituted as defined above.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

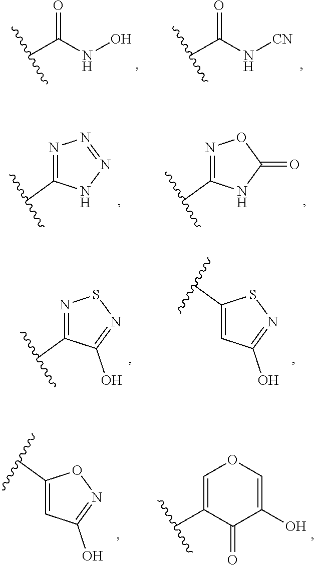

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-TH-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)R^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

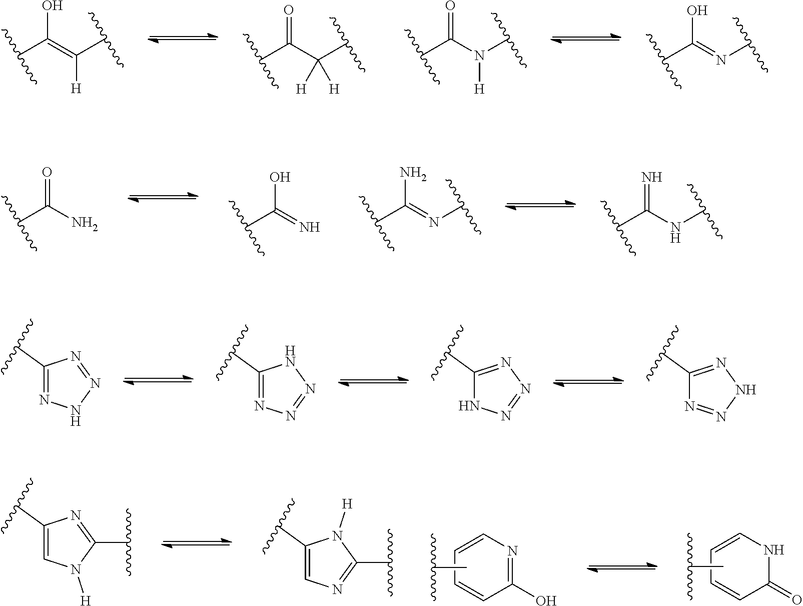

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (2H), tritium (3H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S ³⁴S ³⁵S, ³⁶S, ³⁵Cl, ³⁷Cl, ⁷⁹Br, ⁸¹Br, ¹²⁵I are all contemplated. In some embodiments, isotopic substitution with ¹⁸F is contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

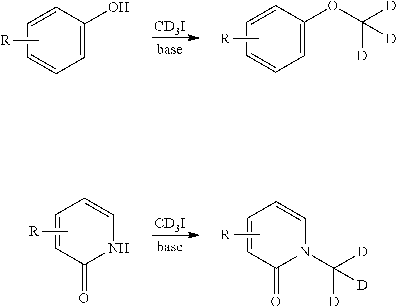

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

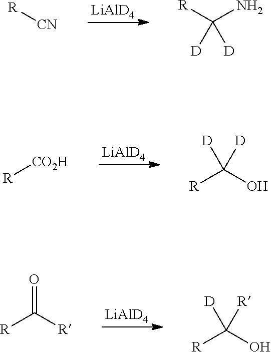

Deuterium gas and palladium catalyst are employed to reduce unsaturated carbon-carbon linkages and to perform a reductive substitution of aryl carbon-halogen bonds as illustrated, by way of example only, in the reaction schemes below.

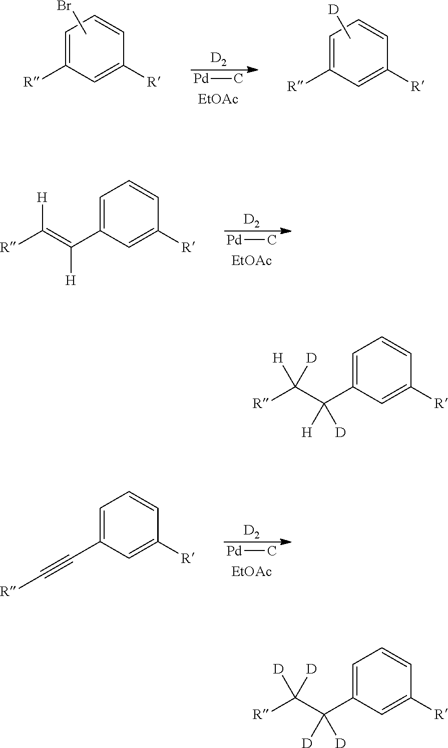

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the heteroaromatic inhibitory compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein optionally exist in either unsolvated as well as solvated forms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

### Ergoline-Derived 5-HT₂ₐ Receptor Agonists Compounds

Lisuride is a dopamine antagonist and a partial agonist for several serotonin receptors. It is an antagonist at the serotonin 5-HT_{2B} receptor (Clin Neuropharmacol. 2006, 29 (2): 80-6). In the brain, 5-HT₂ receptor plays a key role in regulation of cortical function and cognition, appears to be the principal target for the hallucinogenic/psychedelic drugs such as lysergic acid diethylamide (LSD). The 5-HT₂ subfamily of serotonin receptors is composed of three subtypes; namely the 5-HT_{2A}, 5-HT_{2B} and 5-HT_{2C} receptors. All the members of this subfamily couple to the activation of the inositol phosphate and diacyl glycerol pathway via the G-protein, Gq/11. Receptor activities at serotonin receptors, particularly, the 5-HT_{2B} and 5-HT_{2A}/5HT_{2C}, are of specific interest due to their close association with specific adverse events. Compounds that are potent, full agonists at the 5-HT_{2B} receptor have been linked to a risk of retroperitoneal, pleural or cardiac valvular fibrosis. Potent, full agonists at the 5-HT_{2A} receptor pose a risk of psychotic side effects such as hallucinations.

While lisuride has a similar receptor binding profile (5HT_{2A/2C} agonism) to the more well-known and chemically similar ergot alkaloid LSD and inhibits the dorsal raphe serotonergic neurons in a similar fashion to LSD, it lacks the psychedelic effects of its sister compound. Newer findings suggest that the lack of psychedelic action of lisuride arises from the phenomenon of biased agonism (Neurosci. Lett. 2011 493 (3): 76-9; Nature 2008, 452 (7183): 93-7).

Neuropsychiatric diseases, including mood and anxiety disorders, are some of the leading causes of disability worldwide and place an enormous economic burden on society. Approximately one third of patients will not respond to current antidepressant drugs, and those who do will usually require at least two to four weeks of treatment before they experience any beneficial effects. Evidence from a combination of human imaging, postmortem studies, and animal models suggest that atrophy of neurons in the prefrontal cortex (PFC) plays a key role in the pathophysiology of depression and related disorders. These structural changes, such as the retraction of neurites and loss of dendritic spines, can potentially be counteracted by compounds capable of promoting structural and functional neural plasticity. Recently the nonclassical psychedelics has shown remarkable clinical potential as a fast-acting antidepressant and anxiolytic, exhibiting efficacy in treatment-resistant populations. Animal models suggest that its therapeutic effects stem from its ability to promote the growth of dendritic spines, increase the synthesis of synaptic proteins, and strengthen synaptic responses.

Clinical studies have demonstrated the potential for using classical psychedelics to treat a variety of neuropsychiatric disorders including depression, anxiety, addiction, and post-traumatic disorders. However, their therapeutic mechanism of action remains poorly understood, and concerns about safety have severely limited their clinical usefulness.

Psychedelic compounds have the potential to meet the therapeutic needs for a number of indications without the addictiveness and overdose risk of other mind-altering drugs, such as cocaine, heroin, alcohol, methamphetamine, and so forth. The need for new therapies is urgent because addiction, overdose, and suicide deaths have risen throughout the North America and around the world. The problem is further exacerbated by the lack of significant advances in psychiatric drug development, as current treatments are plagued with limited efficacy, significant side effects, and dependency on long time use, which may lead some patients to develop treatment-resistance. Recent academic research effort along with anecdotal reports suggest that psychedelics have promising therapeutic potential (BMC Psychiatry 2018, 18, 245).

Psychedelic compound research has previously been stymied as a result of governmental regulation and societal taboo which has left many unanswered questions regarding the pharmacology and toxicology of psychedelics. There has been renewed interest in the therapeutic potential of psychedelics. For example, psilocybin-assisted psychotherapy has been effective in the treatment of depression and anxiety in cancer patients and also in the treatment of resistant depression (J. Psychopharmacol. 2016, 30, 1181).

Therefore, the future of therapeutic psychedelics research in general holds enormous potential to save lives and meet unmet medical needs throughout the world.

Recently, the serotonin receptor activity profiles (5-HT_{2B} and 5-HT_{2A}) for nine commercialized ergot alkaloids including lisuride were evaluated and the corresponding known risks of cardiac fibrosis and hallucinations reported (American Headache Society 61stAnnual Scientific Meeting July 2019; Philadelphia, PA: Poster 180; see also US 2016/0207920 and WO2018/223065). Lisuride (structure shown below) was found to be a partial agoinst at the 5HT_{2B} with minimal risk of cardiac fibrosis (Toxicol Pathol. 2010, 38 (6):837-48; N Engl J. Med. 2007, 356 (1):6-9; Clin Neuropharmacol. 2006, 29(2):80-6). Furthermore, lisuride was also found to be a potent 5HT_{2A} full agonist with EC₅₀ of 0.3 nM (American Headache Society 61stAnnual Scientific Meeting July 2019; Philadelphia, PA: Poster 180). Compounds that activate the 5-HT_{2A} receptor, such as lysergic acid diethylamide (LSD), act as hallucinogens in humans. However, one notable exception is the LSD congener lisuride, which does not show hallucinogenic effects in humans even though it is a potent 5-HT_{2A} agonists (Psychopharmacology 2010, 208:179-189). As a result, lisuride possesses the highly desired 5HT₂ pharmacological profiles (i.e. 5HT_{2A} agonist, 5HT_{2B} antagonist) that lacks, the psychedelics, hallucinogenic and the cadiac liability providing a safer alternative to potentially treat patients likely to have a positive therapeutic response to a psychedelic agent.

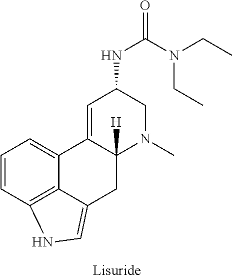

Despites its favorable pharmacological profiles (i.e. 5HT_{2A} agonist, 5HT_{2B} antagonist), lisuride was reported to suffer from poor bioavailability and short in vivo half-life. Considering this, there is urgent need for the development of non-hallucinogenic analogs of psychedelics to treat a variety of brain disorders.

The molecular features that could confer good metabolic and pharmacokinetic characteristic are unpredictable. We have identified key structural features in compounds of Formula (I), Formula (Ia), and Formula (II) that offer improved metabolic properties for the treatment of diseases, disorders or conditions treatable by activating the 5HT_{2A/2C} signaling axis.

The introduction of deuterium and/or fluorine at strategic positions within the Lisuride molecule provided novel derivatives. Deuterium and fluorine modifications can improve a drug's metabolic properties. In this strategy one or more hydrogen atoms in a molecule are replaced with deuterium or fluorine atoms with the aim to slow the CYP-mediated metabolism of a drug or to reduce the formation of undesirable metabolites. Deuterium is a safe, stable, nonradioactive, inexpensive isotope of hydrogen. Deuterium-carbon bonds are stronger than corresponding hydrogen-carbon bonds and in select cases, this increased bond strength will positively impact the absorption, distribution, metabolism, and excretion (ADME) properties of a drug. For example, by decreasing the propensity of a molecule for metabolism by certain enzymes, there is a potential for improved drug efficacy, safety, and/or tolerability. At the same time, because the size and shape of deuterium are essentially identical to those of hydrogen, the corresponding deuterated compound is expected to have similar biological potency compared to the original chemical entity that contains only hydrogen. The effects of deuterium substitution on metabolic stability have been reported for a very small percentage of approved drugs [see, e.g., J Pharm Sci, 1975, 64:367-91; Adv Drug Res, 1985, 14:1-40 (“Foster”); Can J Physiol Pharmacol, 1999, 79-88; Curr Opin Drug Discov Devel, 2006, 9:101-09 (“Fisher”)]. In general, whether or not deuterium modification will affect a compound's metabolic properties is not predictable even when deuterium atoms are incorporated at known sites of metabolism (see, for example, J. Med. Chem., 1991, 34, 2871-76)). One reason for this is that many compounds have multiple sites where metabolism is possible. Therefore, the site(s) where deuterium substitution is required and the extent of deuteration necessary to see an effect on metabolism, if any, will be different for each drug.

In one aspect, provided herein is an ergoline-derived 5-HT₂ₐ receptor agonists compound.

One embodiment provides a compound, or pharmaceutically acceptable salt or solvate thereof, having the structure of Formula (I):

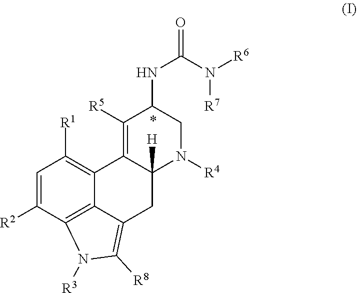

wherein,
R¹ is H, halogen, alkoxy, haloalkoxy (e.g., OCHF₂, OCF₃), or haloalkyl (e.g., CF₃);
R² is H, halogen, alkoxy, haloalkoxy (e.g., OCHF₂, OCF₃), or haloalkyl (e.g., CF₃);
R³ is H, alkyl, or deteuroalkyl;
R⁴ is alkyl or deuteroalkyl;
R⁵ is H or halogen;
R⁶ is optionally substituted C₁₋₆ alkyl or optionally substituted C₁₋₆ alkoxy;
R⁷ is optionally substituted C₁₋₆ alkyl or optionally substituted C₁₋₆ alkoxy;
R⁸ is H or D;
* indicates R or S stereochemistry;
provided that R⁴ is deuteroalkyl or R⁶ is optionally substituted C₁₋₆ alkoxy.
   In some embodiments, R¹ is H, halogen, alkoxy, haloalkoxy, or haloalkyl. In some embodiments, R¹ is H. In some embodiments, R¹ is halogen. In some embodiments, R¹ is alkoxy. In some embodiments, R¹ is haloalkoxy. In some embodiments, R¹ is OCHF₂. In some embodiments, R¹ is OCF₃. In some embodiments, R¹ is haloalkyl. In some embodiments, R¹ is CF₃. In some embodiments, R² is H, halogen, alkoxy, haloalkoxy, or haloalkyl. In some embodiments, R² is H. In some embodiments, R² is halogen. In some embodiments, R² is alkoxy. In some embodiments, R² is haloalkoxy. In some embodiments, R² is OCF₃. In some embodiments, R² is OCHF₂. In some embodiments, R² is haloalkyl. In some embodiments, R² is CF₃. In some embodiments, R³ is H, alkyl, or deteuroalkyl. In some embodiments, R³ is H. In some embodiments, R³ is alkyl. In some embodiments, R³ is deteroalkyl. In some embodiments, R³ is deteuroalkyl. In some embodiments, R³ is cycloalkyl. In some embodiments, R⁴ is alkyl or deuteroalkyl. In some embodiments, R⁴ is alkyl. In some embodiments, R⁴ is deteuroalkyl. In some embodiments, R⁵ is H or halogen. In some embodiments, R⁵ is H. In some embodiments, R⁵ is halogen. In some embodiments, R⁶ is optionally substituted C₁₋₆ alkyl or optionally substituted C₁₋₆ alkoxy. In some embodiments, R⁶ is optionally substituted C₁₋₆ alkyl. In some embodiments, R⁶ is optionally substituted C₁₋₆ alkoxy. In some embodiments, R⁷ is optionally substituted C₁₋₆ alkyl or optionally substituted C₁₋₆ alkoxy. In some embodiments, R⁷ is optionally substituted C₁₋₆ alkyl. In some embodiments, R⁷ is optionally substituted C₁₋₆ alkoxy. In some embodiments, R⁸ is H or D. In some embodiments, R⁸ is H. In some embodiments, R⁸ is D. In some embodiments, in any bond, a hydrogen can be substituted with a deuteurium. In some embodiments, * indicates R or S stereochemistry. In some embodiments, * indicates R stereochemistry. In some embodiments, * indicates S stereochemistry.

In specific embodiments, (a) R⁴ is deuteroalkyl, or (b) R⁶ is optionally substituted C₁₋₆ alkoxy. In specific embodiments, (a) R⁴ is deuteroalkyl, or (b) R⁶ is OCH₃. In specific embodiments, (a) R⁴ is deuteroalkyl, or (b) R⁶ is deuteroalkyl.

In some embodiments, the compound or pharmaceutically acceptable salt or solveate thereof having the structure of Formula (I) is:

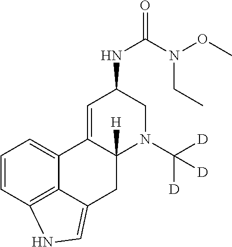

One embodiment provides a compound, or pharmaceutically acceptable salt or solvate thereof, having the structure of Formula (Ia):

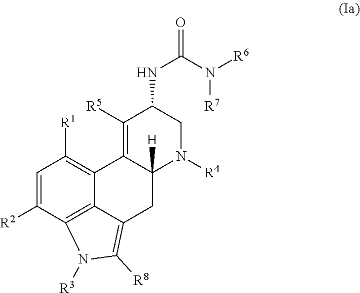

wherein,
R¹ is selected from H, halogen, OMe, CF₃, OCHF₂, and OCF₃;
R² is selected from H, halogen, OMe, CF₃, OCHF₂, and OCF₃;
R³ is selected from H, CH₃ and CD₃;
R⁴ is selected from CH₃ and CD₃;
R⁵ is selected from H or F;
R⁶ is selected from optionally substituted C₁₋₆alkyl, or optionally substituted OC₁-6alkyl;
R⁷ is selected from optionally substituted C₁₋₆alkyl, or optionally substituted OC₁-6alkyl;
R⁸ is selected from H or D;
provided that R¹, R², R³, R⁵, and R⁸ are not H; R⁴ is not CH₃, and R⁶ and R⁷ are not CH₂CH₃.

In some embodiments, R¹ is selected from H, halogen, OMe, CF₃, OCHF₂, and OCF₃. In some embodiments, R¹ is H. In some embodiments, R¹ is H. In some embodiments, R¹ is halogen. In some embodiments, R¹ is OMe. In some embodiments, R¹ is CF₃. In some embodiments, R¹ is OCHF₂. In some embodiments, R¹ is OCF₃. In some embodiments, R² is selected from H, halogen, OMe, CF₃, OCHF₂, and OCF₃. In some embodiments, R² is H. In some embodiments, R² is halogen. In some embodiments, R² is OMe. In some embodiments, R² is CF₃. In some embodiments, R² is OCHF₂. In some embodiments, R² is OCF₃. In some embodiments, R³ is selected from H, CH₃, and CD₃. In some embodiments, R³ is H. In some embodiments, R³ is CH₃. In some embodiments, R³ is CH₃. In some embodiments, R³ is CD₃. In some embodiments, R³ is cycloalkyl. In some embodiments, R⁴ is selected from CH₃ and CD₃. In some embodiments, R⁴ is CH₃. In some embodiments, R⁴ is CD₃. In some embodiments, R⁵ is selected from H or F. In some embodiments, R⁵ is H. In some embodiments, R⁵ is F. In some embodiments, R⁶ is selected from optionally substituted C₁₋₆alkyl, or optionally substituted OC₁-6alkyl. In some embodiments, R⁶ is optionally substituted C₁₋₆ alkyl. In some embodiments, R⁶ is optionally substituted OC₁₋₆ alkyl. In some embodiments, R⁷ is selected from optionally substituted C₁₋₆alkyl, or optionally substituted OC₁₋₆alkyl. In some embodiments, R⁷ is optionally substituted C₁₋₆ alkyl. In some embodiments, R⁷ is optionally substituted OC₁₋₆ alkyl. In some embodiments, R⁸ is selected from H or D. In some embodiments, R⁸ is selected from H or D. In some embodiments, R⁸ is H. In some embodiments, R⁸ is D. In some embodiments, in any bond, a hydrogen (H) can be substituted with a deuteurium (D). In some embodiments, a variable (R) is described herein as being selected from A and B; in such instances the variable (R) is A or B (in other words, the variable (R) is selected from the group consisting of A and B). In some embodiments, R¹, R², R², R³, R⁵, and R⁸ are hydrogen while R⁴ is CD₃. In specific embodiments, R¹, R², R³, R⁵, and R⁸ are not H; R⁴ is not CH₃, and R⁶ and R⁷ are not CH₂CH₃. In some embodiments, the claimed language: provided that R¹, R², R³, R⁵, and R⁸ are not H; R⁴ is not CH₃, and R⁶ and R⁷ are not CH₂CH₃, means: provided that R¹, R², R³, R⁵, and R⁸ are not concurrently H while R⁴ is CH₃ and R⁶ and R⁷ are CH₂CH₃.

One embodiment provides a compound, or pharmaceutically acceptable salt or solvate thereof, having the structure of Formula (Ia):

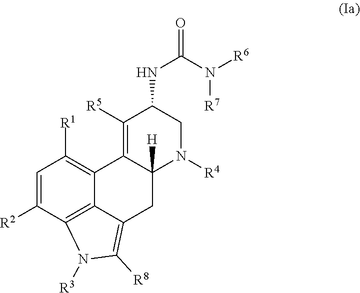

wherein,
R¹ is H, halogen, OMe, CF₃, OCHF₂, or OCF₃;
R² is H, halogen, OMe, CF₃, OCHF₂, or OCF₃;
R³ is H, CH₃ or CD₃;
R⁴ is CH₃ or CD₃;
R⁵ is H or F;
R⁶ is optionally substituted C₁₋₆alkyl, or optionally substituted OC₁₋₆alkyl;
R⁷ is optionally substituted C₁₋₆alkyl, or optionally substituted OC₁₋₆alkyl;
R⁸ is H or D;
provided that R¹, R², R³, R⁵, and R⁸ are not H; R⁴ is not CH₃, and R⁶ and R⁷ are not CH₂CH₃.

In some embodiments, R¹ is H, halogen, OMe, CF₃, OCHF₂, or OCF₃. In some embodiments, R¹ is H. In some embodiments, R¹ is H. In some embodiments, R¹ is halogen. In some embodiments, R¹ is OMe. In some embodiments, R¹ is CF₃. In some embodiments, R¹ is OCHF₂. In some embodiments, R¹ is OCF₃. In some embodiments, R² is H, halogen, OMe, CF₃, OCHF₂, or OCF₃. In some embodiments, R² is H. In some embodiments, R² is halogen. In some embodiments, R² is OMe. In some embodiments, R² is CF₃. In some embodiments, R² is OCHF₂. In some embodiments, R² is OCF₃. In some embodiments, R³ is H, CH₃, or CD₃. In some embodiments, R³ is H. In some embodiments, R³ is CH₃. In some embodiments, R³ is CH₃. In some embodiments, R³ is CD₃. In some embodiments, R³ is cycloalkyl. In some embodiments, R⁴ is CH₃ or CD₃. In some embodiments, R⁴ is CH₃. In some embodiments, R⁴ is CD₃. In some embodiments, R⁵ is H or F. In some embodiments, R⁵ is H. In some embodiments, R⁵ is F. In some embodiments, R⁶ is optionally substituted C₁₋₆alkyl, or optionally substituted OC₁₋₆alkyl. In some embodiments, R⁶ is optionally substituted C₁₋₆ alkyl. In some embodiments, R⁶ is optionally substituted OC₁₋₆ alkyl. In some embodiments, R⁷ is optionally substituted C₁-6alkyl, or optionally substituted OC₁₋₆alkyl. In some embodiments, R⁷ is optionally substituted C₁₋₆ alkyl. In some embodiments, R⁷ is optionally substituted OC₁₋₆ alkyl. In some embodiments, R⁸ is H or D. In some embodiments, R⁸ is selected from H or D. In some embodiments, R⁸ is H. In some embodiments, R⁸ is D. In some embodiments, in any bond, a hydrogen (H) can be substituted with a deuteurium (D). In some embodiments, R¹, R², R², R³, R⁵, and R⁸ are hydrogen while R⁴ is CD₃. In specific embodiments, R¹, R², R³, R⁵, and R⁸ are not H; R⁴ is not CH₃, and R⁶ and R⁷ are not CH₂CH₃. In some embodiments, the claimed language: provided that R¹, R², R³, R⁵, and R⁸ are not H; R⁴ is not CH₃, and R⁶ and R⁷ are not CH₂CH₃, means: R¹, R², R³, R⁵, and R⁸ are not concurrently H while R⁴ is CH₃ and R⁶ and R⁷ are CH₂CH₃.

One embodiment provides a compound, or pharmaceutically acceptable salt or solvate thereof, having the structure of Formula (II):

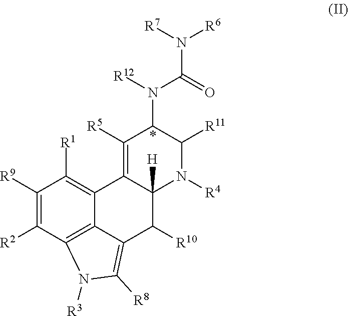

wherein,
R¹ is H, halogen, alkoxy, haloalkoxy (e.g., OCHF₂, OCF₃), or haloalkyl (e.g., CF₃);
R² is H, halogen, alkoxy, haloalkoxy (e.g., OCHF₂, OCF₃), or haloalkyl (e.g., CF₃);
R³ is H, alkyl, or deteuroalkyl;
R⁴ is alkyl or deuteroalkyl;
R⁵ is H or halogen;
R⁶ is optionally substituted C₁₋₆ alkyl or optionally substituted C₁₋₆ alkoxy;
R⁷ is optionally substituted C₁₋₆ alkyl or optionally substituted C₁₋₆ alkoxy;
R⁸ is H or D;
R⁹ is H, halogen, alkoxy, haloalkoxy (e.g., OCHF₂, OCF₃), or haloalkyl (e.g., CF₃);
R¹⁰ is H, D, alkyl, cycloalkyl, or deuteroalkyl;
R¹¹ is H, D, alkyl, cycloalkyl, or deuteroalkyl;
R¹² is H, alkyl, cycloalkyl, or deuteroalkyl;
* indicates R or S stereochemistry;
provided that R⁴ is deuteroalkyl or R⁶ is optionally substituted C₁₋₆ alkoxy.

In some embodiments, R¹ is H, halogen, alkoxy, haloalkoxy (e.g., OCHF₂, OCF₃), or haloalkyl (e.g., CF₃). In some embodiments, R¹ is H. In some embodiments, R¹ is halogen. In some embodiments, R¹ is alkoxy. In some embodiments, R¹ is haloalkoxy. In some embodiments, R¹ is haloalkyl. In some embodiments, R¹ is OCHF₂. In some embodiments, R¹ is OCF₃. In some embodiments, R¹ is CF₃. In some embodiments, R² is H, halogen, alkoxy, haloalkoxy (e.g., OCHF₂, OCF₃), or haloalkyl (e.g., CF₃). In some embodiments, R² is H. In some embodiments, R² is halogen. In some embodiments, R² is alkoxy. In some embodiments, R² is haloalkoxy. In some embodiments, R² is OCHF₂. In some embodiments, R² is OCF₃. In some embodiments, R² is haloalkyl. In some embodiments, R² is CF₃. In some embodiments, R³ is H, alkyl, or deteuroalkyl. In some embodiments, R³ is H. In some embodiments, R³ is alkyl. In some embodiments, R³ is alkyl. In some embodiments, R³ is deuteroalkyl. In some embodiments, R⁴ is alkyl or deuteroalkyl. In some embodiments, R⁴ is alkyl. In some embodiments, R⁴ is deuteroalkyl. In some embodiments, R⁵ is H or halogen. In some embodiments, R⁵ is H. In some embodiments, R⁵ is halogen. In some embodiments, R⁶ is optionally substituted C₁₋₆ alkyl or optionally substituted C₁₋₆ alkoxy. In some embodiments, R⁶ is optionally substituted C₁-6 alkyl. In some embodiments, R⁶ is optionally substituted C₁₋₆ alkoxy. In some embodiments, R⁷ is optionally substituted C₁₋₆ alkyl or optionally substituted C₁₋₆ alkoxy. In some embodiments, R⁷ is optionally substituted C₁₋₆ alkyl. In some embodiments, R⁷ is optionally substituted C₁₋₆ alkoxy. In some embodiments, R⁸ is H or D. In some embodiments, R⁸ is H. In some embodiments, R⁸ is D. In some embodiments, R⁹ is H, halogen, alkoxy, haloalkoxy (e.g., OCHF₂, OCF₃), or haloalkyl (e.g., CF₃). In some embodiments, R⁹ is H. In some embodiments, R⁹ is halogen. In some embodiments, R⁹ is alkoxy. In some embodiments, R⁹ is haloalkoxy. In some embodiments, R⁹ is OCHF₂. In some embodiments, R⁹ is OCF₃. In some embodiments, R⁹ is haloalkyl. In some embodiments, R⁹ is haloalkyl. In some embodiments, R⁹ is CF₃. In some embodiments, R¹⁰ is H, D, alkyl, cycloalkyl, or deuteroalkyl. In some embodiments, R¹⁰ is H. In some embodiments, R¹⁰ is D. In some embodiments, R¹⁰ is cycloalkyl. In some embodiments, R¹⁰ is alkyl. In some embodiments, R¹⁰ is deuteroalkyl. In some embodiments, R¹¹ is H, D, alkyl, cycloalkyl, or deuteroalkyl. In some embodiments, R¹¹ is H. In some embodiments, R¹¹ is D. In some embodiments, R¹¹ is alkyl. In some embodiments, R¹¹ is cycloalkyl. In some embodiments, R¹¹ is deuteroalkyl. In some embodiments, R¹² is H, alkyl, cycloalkyl or deuteroalkyl. In some embodiments, R¹² is H. In some embodiments, R¹² is alkyl. In some embodiments, R¹² is cycloalkyl. In some embodiments, R¹² is deuteroalkyl. In some embodiments, in any bond, a hydrogen can be substituted with a deuteurium. In some embodiments, * indicates R or S steroechemistry. In some embodiments, * indicates R steroeochemistry. In some embodiments, * indicates S stereochemistry.

In specific embodiments, (a) R⁴ is deuteroalkyl, or (b) R⁶ is optionally substituted C₁₋₆ alkoxy. In specific embodiments, (a) R⁴ is deuteroalkyl, or (b) R⁶ is OCH₃. In specific embodiments, (a) R⁴ is deuteroalkyl, or (b) R⁶ is deuteroalkyl.

In some embodiments, the ergoline-derived 5-HT₂ₐ receptor agonists compound as described herein has a structure provided in Table 1.

TABLE 1

| Synthetic | |
|---|---|
| Chemistry | |
| Example | Compound Structure |
| 1 | 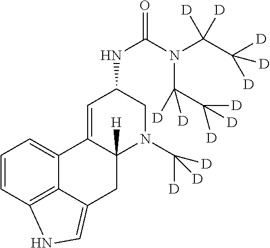 |
| 2 | 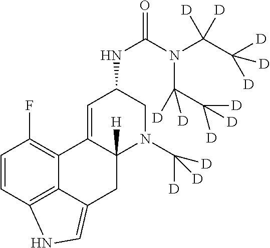 |
| 3 | 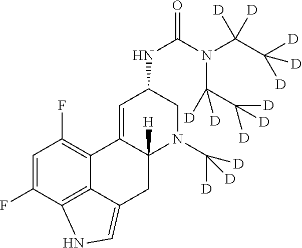 |
| 4 | 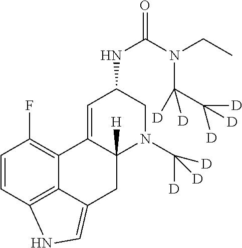 |
| 5 | 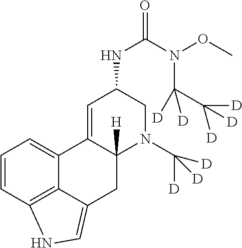 |
| 6a | 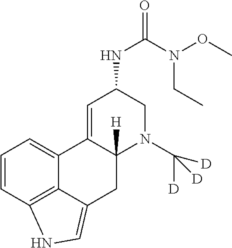 |
| 6b | 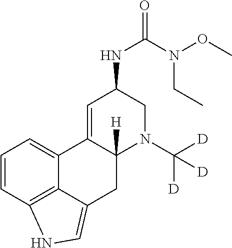 |
| 7 | 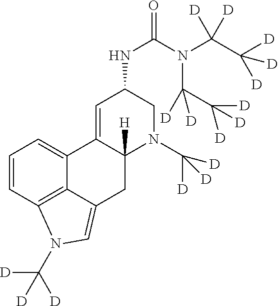 |
| 8 | 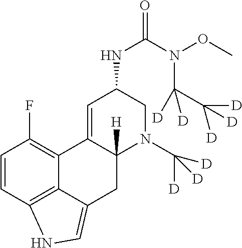 |
| 9 | 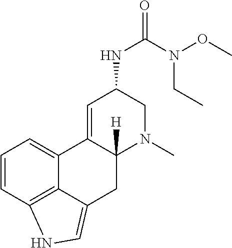 |
| 10 | 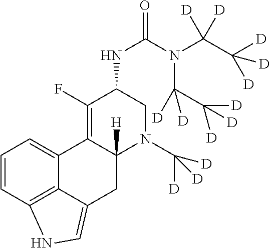 |
| 11 | 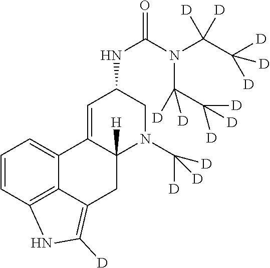 |
| 12 | 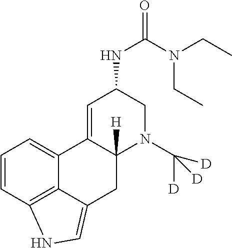 |

### Preparation of Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Comwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen A G (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference useful for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

The compounds of Formula (I), (Ia), and (II) generally can be prepared according to the processes illustrated in the Schemes below. In the structural formulae shown below the variables are as defined in Formula (I), (Ia), or (II) unless otherwise stated.

In some embodiments, the compounds of Formula (I), wherein R³═H, R⁵═H and R⁸═H are prepared as shown in Scheme 1 and Scheme 2.

The corresponding 3-(substituted-1H-indol-3-yl) propanoic acid (A, commercially available) which is treated with pivalolyl chloride to give intermediate B. Acyl chloride formation and subsequent intramolecular acylation affords the intermediate C (see WO2016052697) as shown in Scheme 1.

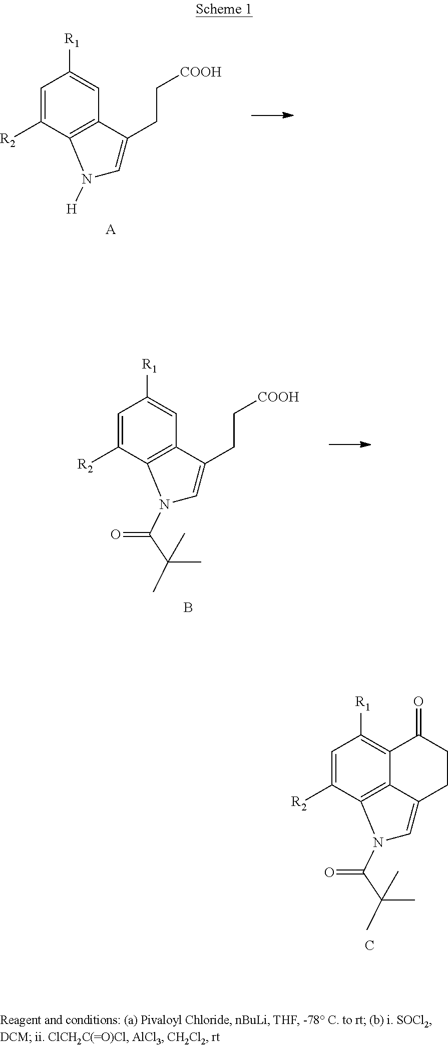

The pivaloyl derivative C was then subjected to the synthetic sequences outlined in the following references and as shown in Scheme 2: Journal of the American Chemical Society 1956, 78, 3087-3114; Journal of Organic Chemistry 2004, 69(18), 5993-6000; Tetrahedron 2003, 59(24), 4281-4286; WO 2016052697.

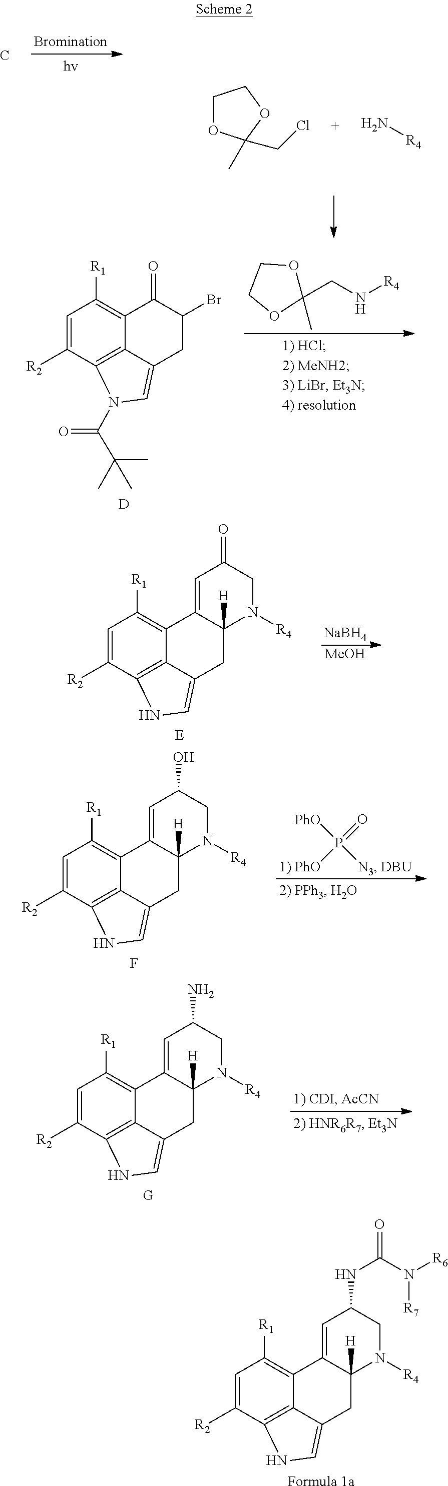

In some embodiments, compounds of Formula (I), (Ia), or (II) in which R³═H, R⁵═H and R⁸=D can be prepared through a sequence of halogenation (Bromination or Iodination, see WO2018223065), metal halogen-exchange and quenching with D₂O or CD₃OD (Scheme 3).

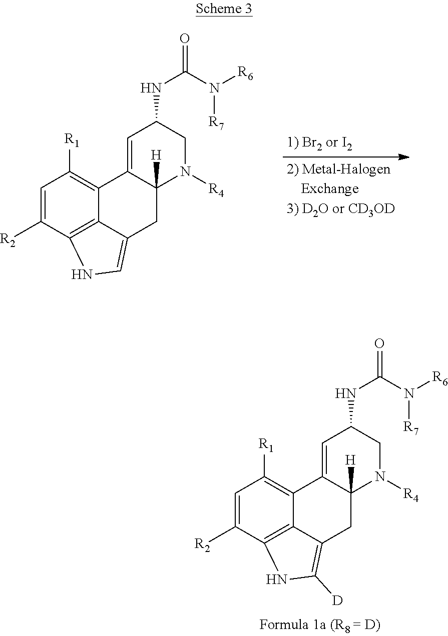

In some embodiments, compounds of Formula (I), (Ia), or (II) wherein R³=Me or CD₃, R⁵═H and R⁸═H can be prepared as shown in Scheme 4.

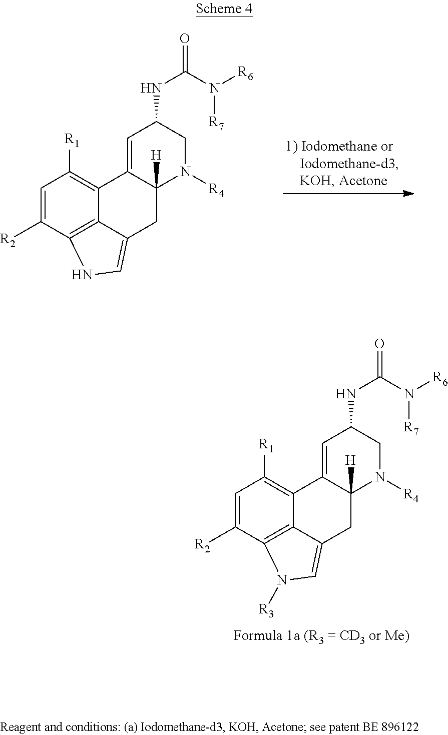

In an alternate embodiment, compounds of Formula (I), (Ia), or (II) wherein R³═H, R⁵ ═F, R⁸═H can be via intermediate J prepared as shown in Scheme 5. Intermediate J is then transformed to the desired targets following similar synthetic sequences as in Scheme 1.

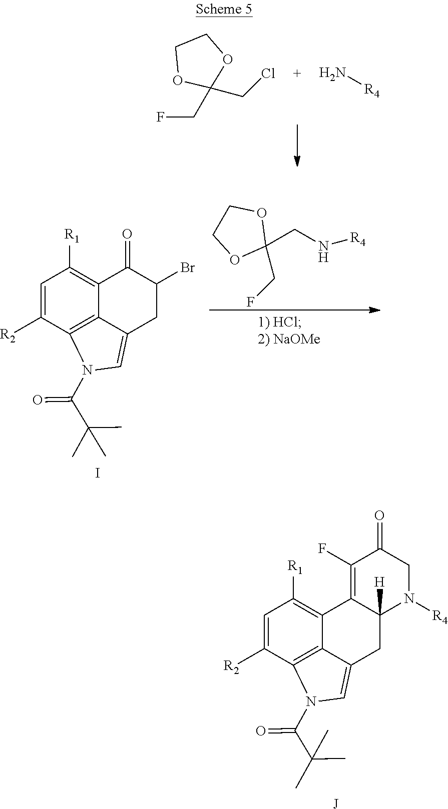

In some embodiments, compounds comprising intermediates for lisuride derivatives, such as intermediate S, can be prepared via intermediate K and intermediate L, as shown in Scheme 6.

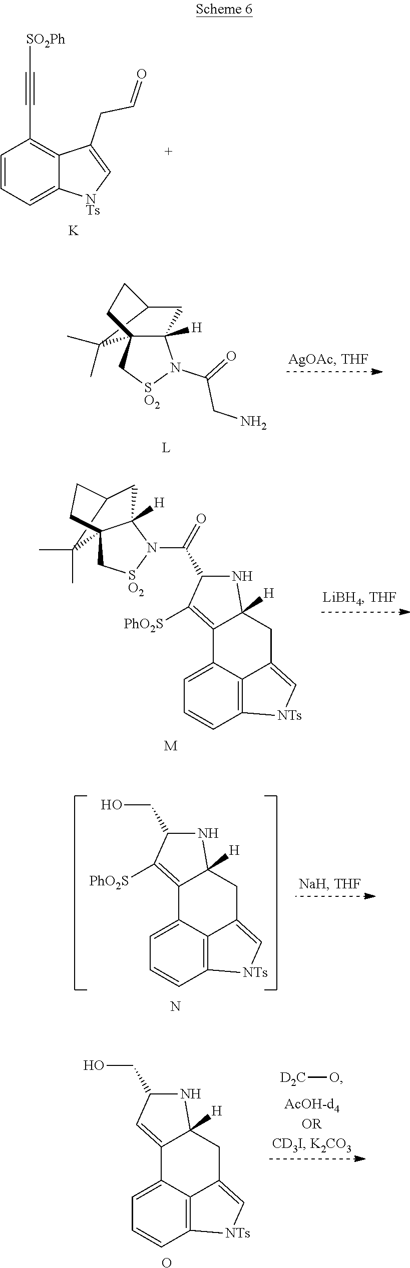

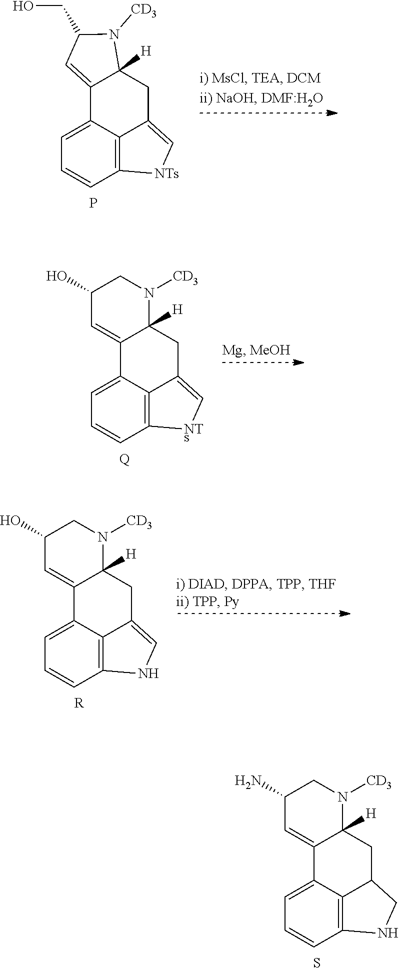

In some embodiments, intermediate K can be prepared as shown in Scheme 7.

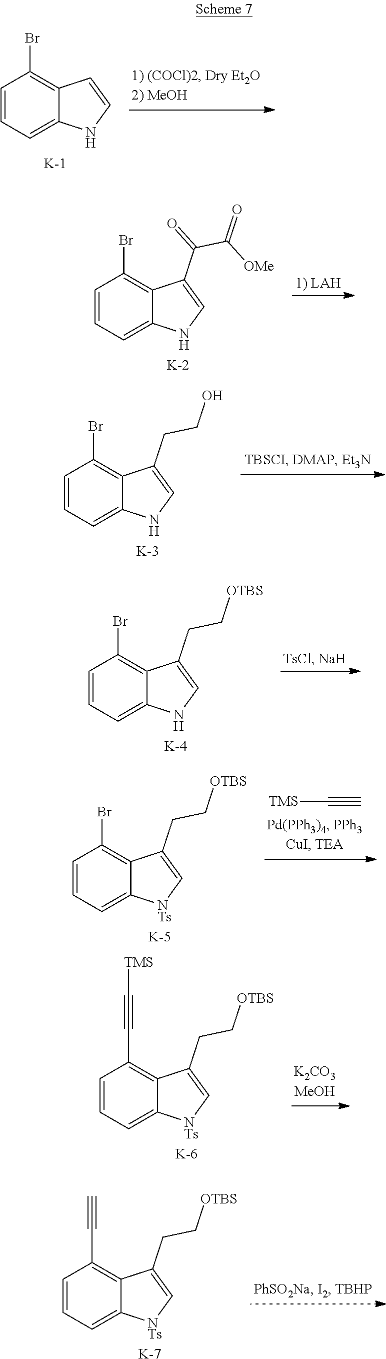

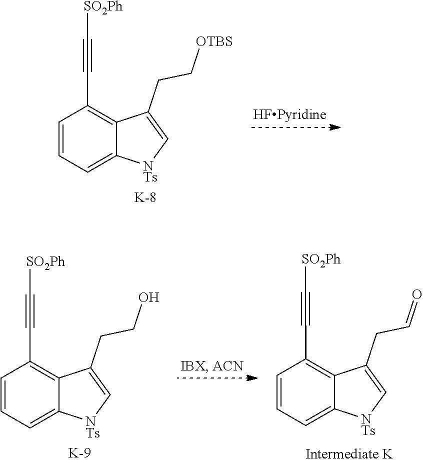

In some embodiments, intermediate L can be prepared as shown in Scheme 8.

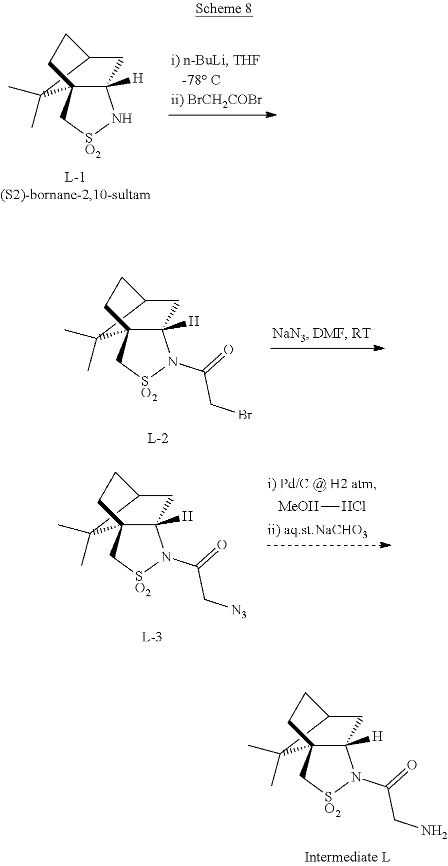

Generally, the reactions described above are performed in a suitable inert organic solvent and at temperatures and for times that will optimize the yield of the desired compounds.

Examples of suitable inert organic solvents include, but are not limited to, dimethylformamide (DMF), dioxane, methylene chloride, chloroform, tetrahydrofuran (THF), toluene, and the like.

### Pharmaceutical Compositions

In certain embodiments, the ergoline-derived 5-HT₂ₐ receptor agonists compound described herein is administered as a pure chemical. In other embodiments, the ergoline-derived 5-HT₂ₐ receptor agonists compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice ofPharmacy (Gennaro, 21^{st} Ed. Mack Pub. Co., Easton, PA (2005)).

Provided herein is a pharmaceutical composition comprising at least one ergoline-derived 5-HT₂ₐ receptor agonists compound as described herein, or a stereoisomer, pharmaceutically acceptable salt, hydrate, or solvate thereof, together with one or more pharmaceutically acceptable carriers. The carrier(s) (or excipient(s)) is acceptable or suitable if the carrier is compatible with the other ingredients of the composition and not deleterious to the recipient (i.e., the subject or the patient) of the composition.

One embodiment provides a pharmaceutical composition comprising a pharmaceutically acceptable excipient and a compound of Formula (I), or a pharmaceutically acceptable salt or solvate thereof. One embodiment provides a pharmaceutical composition comprising a pharmaceutical acceptable excipient and a compound of Formula (Ia), or a pharmaceutically acceptable salt or solvate thereof. One embodiment provides a pharmaceutical composition comprising a pharmaceutically acceptable excipient and a compound of Formula (II).

One embodiment provides a method of preparing a pharmaceutical composition comprising mixing a compound of Formula (I), or a pharmaceutically acceptable salt or solvate thereof, and a pharmaceutically acceptable carrier. One embodiment provides a method of preparing a pharmaceutical composition comprising mixing a compound of Formula (Ia), or a pharmaceutically acceptable salt or solvate theoreof, and a pharmaceutically acceptable carrier. One embodiment provides a method of preparing a pharmaceutical composition comprising mixing a compound of Formula (II), or a pharmaceutically acceptable salt or solvate theoreof, and a pharmaceutically acceptable carrier.

In certain embodiments, the ergoline-derived 5-HT₂ₐ receptor agonists compound as described by Formula (I), or a pharmaceutically acceptable salt or solvate thereof, is substantially pure, in that it contains less than about 5%, or less than about 1%, or less than about 0.1%, of other organic small molecules, such as unreacted intermediates or synthesis by-products that are created, for example, in one or more of the steps of a synthesis method. In certain embodiments, the ergoline-derived 5-HT₂ₐ receptor agonists compound as described by Formula (Ia), or a pharmaceutically acceptable salt or solvate thereof, is substantially pure, in that it contains less than about 5%, or less than about 1%, or less than about 0.1%, of other organic small molecules, such as unreacted intermediates or synthesis by-products that are created, for example, in one or more of the steps of a synthesis method. In certain embodiments, the ergoline-derived 5-HT₂a receptor agonists compound as described by Formula (II), or a pharmaceutically acceptable salt or solvate thereof, is substantially pure, in that it contains less than about 5%, or less than about 1%, or less than about 0.1% of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

In some embodiments, the ergoline-derived 5-HT₂ₐ receptor agonists compound as described by Formula (I), or pharmaceutically acceptable salt or solvate thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.
In some embodiments, the ergoline-derived 5-HT₂ₐ receptor agonists compound as described by Formula (Ia), or pharmaceutically acceptable salt or solvate thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.
In some embodiments, the ergoline-derived 5-HT₂ₐ receptor agonists compound as described by Formula (II), or pharmaceutically acceptable salt or solvate thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising at least one ergoline-derived 5-HT₂ₐ receptor agonists compound as described herein differs depending upon the subject or patient's (e.g., human) condition. In some embodiments, such factors include general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### Methods of Treatment

One embodiment provides a compound of Formula (I), or a pharmaceutically acceptable salt or solvate thereof, for use in a method of treatment of the human or animal body. One embodiment provides a compound of Formula (Ia), or a pharmaceutically acceptable salt or solvate thereof, for use in a method of treatment of the human or animal body. One embodiment provides a compound of Formula (II), or a pharmaceutically acceptable salt or solvate thereof, for use in a method of treatment of the human or animal body.

One embodiment provides a compound of Formula (I), Formula (Ia), Formula (II), or a pharmaceutically acceptable salt or solvate thereof, for use in a method of treatment of a disease or disorder mediated by the 5-HT₂ receptor. In some embodiments, the disease or disorder is is mediated by activating the 5-HT_{2A/2C} receptor signaling axis. In some embodiments, the disease, disorder or condition that is treatable by activating the 5-HT_{2A/2C} receptor, is a CNS disorder. In some embodiments, the treatment comprises administration of an amount of at least one compounds described herein that is effective to ameliorate at least one symptom of a brain disorder, for example, improvement in mental or physical well-being in the subject (e.g., by treating stress, anxiety, addiction, depression, compulsive behavior, by promoting weight loss, by improving mood, by treating or preventing a condition (e.g. psychological disorder), or by enhancing performance.

A “5-HT_{2A/2C} receptor-mediated disorder”, as used herein, is a disorder in which there is believed to be involvement of the pathway controlled by the 5-HT_{2A/2C} receptor and which is ameliorated by treatment with an agonist of the 5-HT_{2A/2C} receptor. 5-HT_{2A/2C} receptor-mediated disorders include a depressive disorder, an anxiety disorder, including panic attack, agoraphobia, and specific or social phobia, bipolar disorder, post-traumatic stress, an eating disorder, obesity, a gastro-intestinal disorder, alcoholism, drug addiction, schizophrenia, a psychotic disorder, a sleep disorder, including sleep apnea, migraine, sexual dysfunction, a central nervous system disorder, including trauma, stroke and spinal cord injury, a cardio-vascular disorder, diabetes insipidus, obsessive.

Provided herein is the method wherein the pharmaceutical composition is administered orally. Provided herein is the method wherein the pharmaceutical composition is administered by injection.

Other embodiments and uses will be apparent to one skilled in the art in light of the present disclosures. The following examples are provided merely as illustrative of various embodiments and shall not be construed to limit the invention in any way.

### EXAMPLES

### I. Chemical Synthesis

In some embodiments, the ergoline-derived 5-HT₂ₐ receptor agonists compound disclosed herein are synthesized according to the following examples.

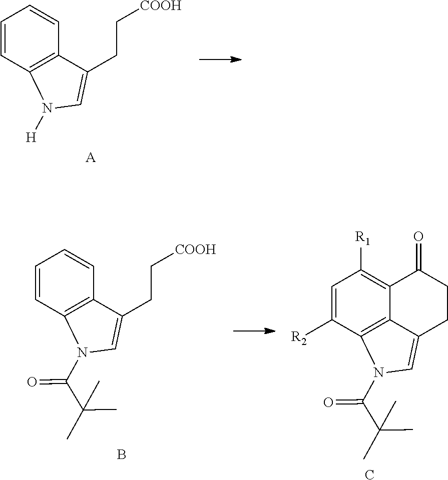

### (a) Synthesis of 3-(1-pivaloyl-1H-indol-3-yl) propanoic acid

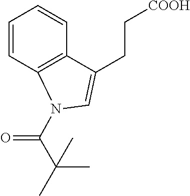 [SMILES: CC(C)(C)C(=O)n1cc(CCC(=O)O)c2ccccc21]

To a solution of 3-(Indol-3-yl) propanoic acid (2.5 g, 13 mmol) in THF (75 mL) at −78° C. under Ar was added a 1.6 M solution of BuLi in hexane (16.5 mL, 26 mmol). After 5 min, trimethylacetyl chloride (1.6 mL, 13 mmol) was added to the mixture which was then stirred for 15 min at −78° C., for 15 min at −50° C. and for 15 min at −20° C. The reaction was quenched with sat. aq NH₄Cl solution and the mixture was extracted with EtOAc (3×100 mL). The combined organic extracts were washed with brine, dried (MgSO₄) and evaporated under reduced pressure. The residue was chromatographed using EtOAc and hexane on a silica gel column. Colorless prisms yield 3.3 g, 91%. LCMS: m/z=273 (M+). In a similar manner the following compound was synthesized.

| | Name | Yield & Mass |
|---|---|---|
| 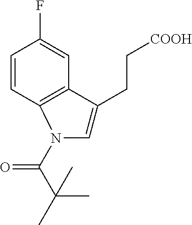 [SMILES: CC(C)(C)C(=O)n1cc(CCC(=O)O)c2cc(F)ccc21] | 3-(5-fluoro-1-pivaloy1-1H-indol- 3-y1) propanoic acid Exact Mass: 291.13 | 75% yield LCMS [M]⁺ 291 |
| 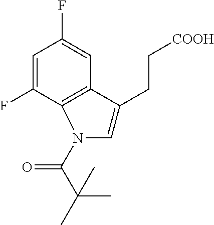 [SMILES: CC(C)(C)C(=O)n1cc(CCC(=O)O)c2cc(F)cc(F)c21] | 3-(5,7-difluoro-1-pivaloy1-1H- indol-3-y1) propanoic acid Exact Mass: 309.12 | 85% yield LCMS [M]⁺ 309 |

### (b) Synthesis of 1-pivaloyl-3,4-dihydrobenzo[cd]indol-5(1H)-one

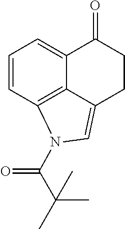 [SMILES: CC(C)(C)C(=O)n1cc2c3c(cccc31)C(=O)CC2]

3-(1-pivaloyl-1H-indol-3-yl) propanoic acid (1.5 mmol) was treated with SOCl₂ (0.56 mL, 7.5 mmol) for 20 min at r.t. and then SOCl₂ was evaporated under reduced pressure. The acid chloride was dissolved in 1,2-dichloroethane (15 mL) and to this was added a solution of AlCl₃ (0.20 g, 6.0 mmol) and propionyl chloride (0.52 m, 6.0 mmol) or chloroacetyl chloride (0.48 m, 6.0 mmol) in 1,2-dichloroethane (10 mL). Then, the mixture was stirred for 1-36 h at 15° C. The reaction mixture was poured into the mixture of ice and CH₂Cl₂ (15 mL) and the organic layer was separated. Aqueous layer was extracted with CH₂Cl₂ (2×15 mL). The combined organic layers were washed with H₂O (3×30 mL), dried (Na₂SO₄) and the solvent was evaporated under reduced pressure. The product 5, 6, 8 or 9 was isolated by silica gel chromatography (EtOAc/hexane). 1-Trimethylacetyl-3,4-dihydrobenz [c, d 1 indol-5(1H)-one, yield 83%. LCMS: m/z=255 (M+).

| | Name | Yield & Mass |
|---|---|---|
| 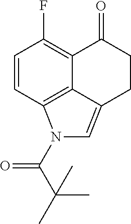 [SMILES: CC(C)(C)C(=O)n1cc2c3c(c(F)ccc31)C(=O)CC2] | 6-fluoro-1-pivaloyl-3,4- dihydrobenzo[cd]indol- 5(1H)-one Exact Mass: 273.12 | 75% yield LCMS [M]+ 273 |
| 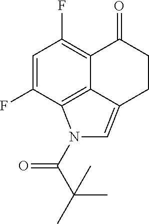 [SMILES: CC(C)(C)C(=O)n1cc2c3c(c(F)cc(F)c31)C(=O)CC2] | 6,8-difluoro-1-pivaloyl- 3,4- dihydrobenzo[cd]indol- 5(1H)-one Exact Mass: 291.11 | 55% yield LCMS [M]+ 292 |

### (c) Synthesis of 4-bromo-1-pivaloyl-3,4-dihydrobenzo[cd]indol-5(1H)-one

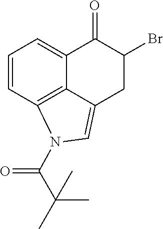 [SMILES: CC(C)(C)C(=O)n1cc2c3c(cccc31)C(=O)C(Br)C2]

A solution of 0.304 g (1.1 mmoles) of 1-benzoyl-5-keto-1,2,2a,3,4,5-hexahydrobenz[cd]indole in 5 ml of glacial acetic acid was warmed to 40°. While the reaction was illuminated with a 250-watt bulb, 0.352 g (1.1 mmoles) of pyridine hydrobromide perbromide was added in portions during 5 minutes with shaking. The solution was warmed to 600 and kept at 55-60° for 0.5 hour. The mixture was treated with carbon and concentrated to small volume in vacuo. The residue was taken up in 20 ml of chloroform and the solution was washed several times with water dried over magnesium sulfate and concentrated in vacuo. The residue was crystallized from 5 ml of 1:1 acetic acid-ether; yield 0.270 g (69%). MS (m/z): 334 (M+H)+.

In a similar manner the following compound was synthesized.

| | Name | Yield & Mass |
|---|---|---|
| 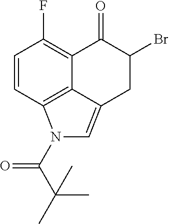 [SMILES: CC(C)(C)C(=O)n1cc2c3c(c(F)ccc31)C(=O)C(Br)C2] | 4-bromo-6-fluoro-1-pivaloy1-3,4- dihydrobenzo[cd]indol-5(1H)-one Exact Mass: 351.03 | 55% yield LCMS [M]+ 351 |
| 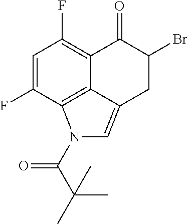 [SMILES: CC(C)(C)C(=O)n1cc2c3c(c(F)cc(F)c31)C(=O)C(Br)C2] | 4-bromo-6,8-difluoro-1-pivaloyl- 3,4-dihydrobenzo[cd]indol-5(1H)- one Exact Mass: 369.02 | 60% yield LCMS [M]+ 369 |

### (d) Synthesis of (R)-7-methyl-6,6a,7,8-tetrahydroindolo[4,3-fg] quinolin-9(4H)-one Exact Mass: 238.11

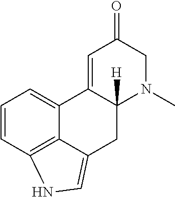

To a solution of 4-bromo-1-pivaloyl-3,4-dihydrobenzo[cd]indol-5(1H)-one (1.12 g, 3.35 mmol) in dry toluene (35 mL) was added amine N-methyl-1-(2-methyl-1,3-dioxolan-2-yl) methanamine (1.1 g, 8.3 mmol) in toluene (3.5 mL) at room temperature and the solution was stirred for 48 h. The precipitate formed was filtered off and washed with toluene and the combined filtrate was evaporated in vacuo. Purification by chromatography (eluent: EtOAc/hexane, 2:1) afforded 4-(methyl((2-methyl-1,3-dioxolan-2-yl) methyl) amino)-1-pivaloyl-3,4-dihydrobenzo[cd]indol-5(1H)-one (0.43 g, 35%) as a colorless oil.

Methylamine gas was then introduced into a solution of 4-(methyl((2-methyl-1,3-dioxolan-2-yl) methyl) amino)-1-pivaloyl-3,4-dihydrobenzo[cd]indol-5(1H)-one (0.5 g, 1.3 mmol) in benzene (50 mL) at 10-15° C. for about 1 h. The mixture was washed with water and brine and dried. The crude mixture of targeted intermediate 4-(methyl((2-methyl-1,3-dioxolan-2-yl) methyl) amino)-3,4-dihydrobenzo[cd]indol-5(1H)-one (0.312 g, 80%) was dissolved in aq. HCl solution (6 M, 100 mL) and stirred at 37° C. for 1 h, then cooled in an ice bath. The mixture was mixed with CHCl₃ (0.5 L), and the pH was adjusted to −7 with aq. NaOH solution (5 M). After the phases were separated, the aqueous phase was washed with CHCl₃ (2×100 mL). The combined organic phase was washed with brine (100 mL) and dried. An aliquot part was evaporated (bath: 25-30° C.) in vacuo and the residue was crystallized (ether/hexane, 1:1) to yield 4-(methyl (2-oxopropyl) amino)-3,4-dihydrobenzo[cd]indol-5(1H)-one as a pale brown solid which was use directly in the next step.

To a solution of LiBr (2.82 g, 32.5 mmol) in THF (40 mL) at 0-5° C. were added the solution of 4-(methyl(2-oxopropyl) amino)-3,4-dihydrobenzo[cd]indol-5(1H)-one in CHCl₃, obtained after extraction and evaporation to about 100 mL, and TEA (2.82 g, 28 mmol) at 0-5° C. The mixture was stirred at the above temperature for 12 h, then evaporated (bath: 30° C.). The residue was treated with n-hexane to remove TEA. The obtained oil was purified by chromatography (eluent: CHCl₃/MeOH, 10:1) to afford a semisolid product, which was crystallized (EtOAc/hexane, 1:1, 20 mL) to yield 0.758 g (60%) of (RS)-7-methyl-6,6a,7,8-tetrahydroindolo[4,3-fg] quinolin-9(4H)-one as pale-yellow crystals.

Resolution to the (+) enantiomer. To a solution of racemic (RS)-7-methyl-6,6a,7,8-tetrahydroindolo[4,3-fg] quinolin-9(4H)-one (595 mg, 2.5 mmol) in a mixture of acetonitrile and water (1:1, 25 mL) at 60° C. was added (−)-dibenzoyl-L-tartaric acid (895 mg, 2.5 mmol) in the same mixture of solvents (12.5 mL). The mixture was stirred for 10-15 min at the above temperature, then cooled to room temperature while being stirred for about an additional 0.5 h. The mixture was kept in a refrigerator overnight. The precipitated crystals were filtered off and washed with the above solvent mixture (5 mL) to yield 585 mg (79%) of salt. [R]D+271 (c 0.265, MeOH). This salt (515 mg, 0.864 mmol) was suspended in a mixture of CHCl₃ (200 mL) and water (30 mL) at 0-5° C. and the pH was adjusted to 9 with aq NaOH solution (1 M, 2 mL). After the phases were separated, the aqueous phase was washed with CHCl₃ (250 mL). The combined organic phases were washed with water, dried, and evaporated. The residue was crystallized (hexane/ether, 1:1, 10 mL) to yield (R)-7-methyl-6,6a,7,8-tetrahydroindolo[4,3-fg]quinolin-9(4H)-one (226 mg, 38%) as a yellow crystal. Mp: 165-169° C. [R]D+686 (c 0.5, MeOH). LCMS [M]+238.

In a similar manner using N-((2-methyl-1,3-dioxolan-2-yl) methyl) methan-d3-amine the following compounds were prepared.

| | Name | Yield & Mass |
|---|---|---|
| 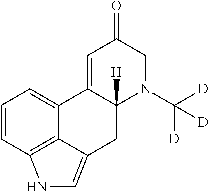 | (R)-7-(methyl-d3)-6,6a,7,8- tetrahydroindolo[4,3-fg] quinolin- 9(4H)-one Exact Mass: 241.13 | 30% yield LCMS [M]+ 241 |
| 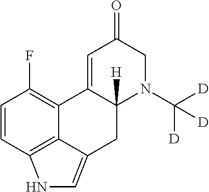 | (R)-1-fluoro-7-(methyl-d3)- 6,6a,7,8-tetrahydroindolo[4,3-fg] quinolin-9(4H)-one Exact Mass: 259.12 | 40% yield LCMS [M]+ 259 |
| 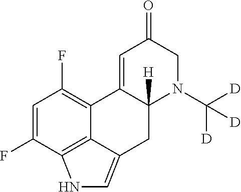 | (R)-1,3-difluoro-7-(methyl-d3)- 6,6a,7,8-tetrahydroindolo[4,3-fg] quinolin-9(4H)-one Exact Mass: 277.11 | 36% yield LCMS [M]+ 277 |

### (e) Synthesis of (6aR,9S)-7-methyl-4,6,6a,7,8,9-hexahydroindolo[4,3-fg] quinolin-9-ol

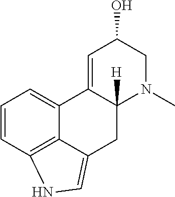

(R)-7-methyl-6,6a,7,8-tetrahydroindolo[4,3-fg] quinolin-9(4H)-one (1 g) was added to a mixture of 150 mL of methanol and 10 mL of water. Sodium borohydride, 0.15 g was added, and the reaction was allowed to proceed at room temperature for about two hours. The solution was then concentrated to small volume, and a mixture of 15 mL of concentrated hydrochloric acid and 60 mL of water was added. The hydrochloride which separated on cooling was filtered and washed with methanol to give 0.9 g (79%). A sample was recrystallized from dilute ethanol; to yield 60%. MS (m/z): 241 (M+H)+.

In a similar manner the following compounds were prepared.

| | Name | Yield & Mass |
|---|---|---|
| 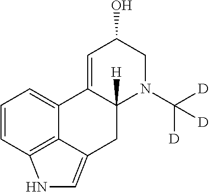 | (6aR,9S)-7-(methyl-d3)- 4,6,6a,7,8,9-hexahydroindolo[4,3- fg] quinolin-9-ol Exact Mass: 243.15 | 75% yield LCMS [M]+ 243 |
| 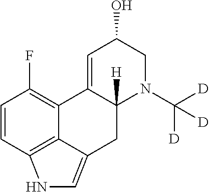 | (6aR,9S)-1-fluoro-7-(methyl-d3)- 4,6,6a,7,8,9-hexahydroindolo[4,3- fg] quinolin-9-ol Exact Mass: 261.14 | 60% yield LCMS [M]+ 261 |
| 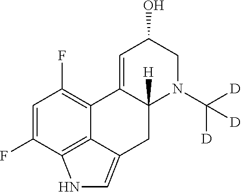 | (6aR,9S)-1,3-difluoro-7-(methyl- d3)-4,6,6a,7,8,9- hexahydroindolo[4,3-fg] quinolin- 9-ol Exact Mass: 279.13 | 64% yield LCMS [M]+ 279 |

### (f) Synthesis of (6aR,9S)-7-methyl-4,6,6a,7,8,9-hexahydroindolo[4,3-fg] quinolin-9-amine

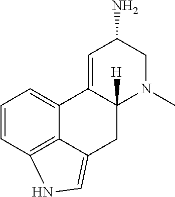

To a solution of (6aR,9S)-7-methyl-4,6,6a,7,8,9-hexahydroindolo[4,3-fg] quinolin-9-ol in Et₂O at 0° C. was added diphenylphosphoryl azide (1.2 eq) followed by slow addition of DBU (1.2 eq). After stirring the reaction mixture overnight, it was diluted with toluene and washed with H₂O (3×50 mL), brine (1×50 mL), dried over MgSO₄ and concentrated. Purification by column chromatography using hexane: EtOAc (4:1) as eluant gave the desired azido compound. This azido compound was then dissolved in THF: H₂O (3:1), Ph₃P (1.1 eq) was added, followed by KOH (1.0 eq). The resulting mixture was stirred overnight. The reaction mixture was then diluted with H₂O and slowly acidified with HCl, and the aqueous layer was washed with Et₂O (3×50 mL). The aqueous layer was then basified with NaOH (pH 14) and extracted with Et₂O (3×50 mL). The combined organic extracts were washed with H₂O (1×25 mL), brine (1×25 mL), dried over K₂CO₃ and concentrated to give the desired (6aR,9S)-7-methyl-4,6,6a,7,8,9-hexahydroindolo[4,3-fg] quinolin-9-amine. Yield 60%. MS (m/z): 240 (M+H)+.

In a similar manner the following compounds were prepared.

| | Name | Yield & Mass |
|---|---|---|
| 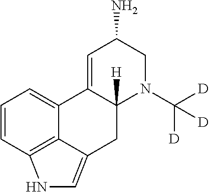 | (6aR,9S)-7-(methyl-d3)- 4,6,6a,7,8,9-hexahydroindolo[4,3- fgquinolin-9-amine Exact Mass: 242.16 | 73% yield LCMS [M]+ 242 |
| 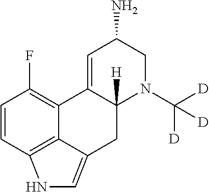 | (6aR,9S)-1-fluoro-7-(methyl-d3)- 4,6,6a,7,8,9-hexahydroindolo[4,3- fgquinolin-9-amine Exact Mass: 260.15 | 51% yield LCMS [M]+ 260 |
| 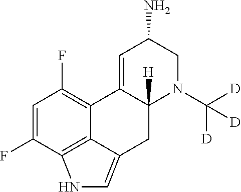 | (6aR,9S)-1,3-difluoro-7-(methyl- d3)-4,6,6a,7,8,9- hexahydroindolo[4,3-fg]quinolin- 9-amine Exact Mass: 278.14 | 62% yield LCMS [M]+ 278 |

### (g) Synthesis—of (R)-10-fluoro-7-methy-d3)-6,6a,78-tetrahydroindolo[4,3-fg] quinolin-9(4H)-one

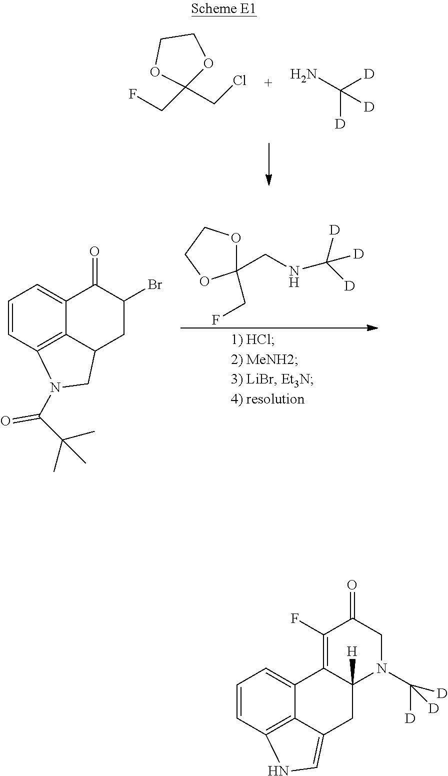 [0195] Journal of the Chemical Society (1958), 2259-62; Helvetica Chimica Acta (1958), 41, 560-73.

| | Name | Yield & Mass |
|---|---|---|
| 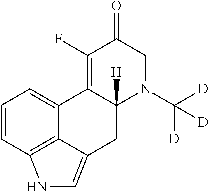 | (R)-10-fluoro-7-(methyl-d3)- 6,6a,7,8-tetrahydroindolo[4,3-fg] quinolin-9(4H)-one Exact Mass: 259.12 | 33% yield LCMS [M]+ 259 |

Synthesis of the N-Ethyl-d5-O-methyl-hydroxylamine derivative Example and Example 8 required synthesis of the deuterated amine shown in Scheme E2 (see references US20100029670 and US 20160185777).

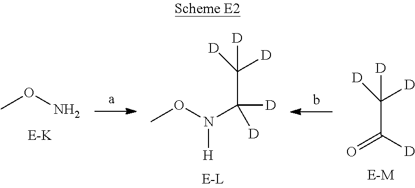

Reagent and conditions: (a) i. ethyl chloroformate, DCM, Et3N −40° C.; ii. NaH, Bromoethane-d5 (commercial)), DMF; 0° C.; then 80° C.; iii. KOH, EtOH-H₂O (b)O-methylhydroxylamine hydrochloride, AcONa, CD₃OD, 15° C.; ii. NaBD₄; 15° C.

### (h) Synthesis of 1,1-bis(ethyl-d5)-3-((6aR,9S)-7-(methyl-d3)-4,6,6a,7,8,9-hexahydroindolo[4,3-fg]quinolin-9-yl)urea, (Example 1)

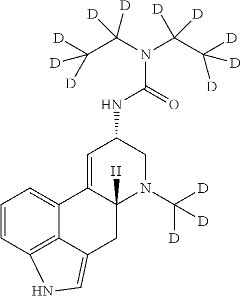

(6aR,9S)-7-methyl-4,6,6a,7,8,9-hexahydroindolo[4,3-fg] quinolin-9-amine (1.0 eq) was mixed with diimidazole carbonyl (1.0 eq) in acetonitrile at room temperature, and then diisopropylethyl amine (2.0 eq) was added. The resulting reaction mixture was stirred for 14 hours. The amine (1.0 eq) in THF was then added and the resulting mixture was stirred for another 14 hours. The reaction mixture was diluted with EtOAc and washed with H₂O (3×75 mL), then concentrated. Chromatography (gradient solvent system, from 50% EtOAc/hexanes to 10% Methanol/EtOAc) or recrystallization in CH₃CN gave the desired title compounds; Yield 80%. MS (m/z): 351 (M+H)+.

In a similar manner, the following compounds were prepared

| | | | Yield & |
|---|---|---|---|
| Example # | Structures | Name | Mass |
| Example 2 | 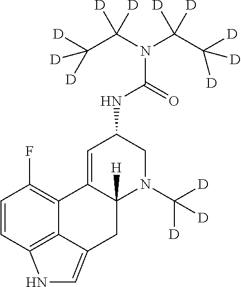 | 1,1-bis(ethyl-d5)-3- ((6aR,9S)-1-fluoro-7- (methyl-d3)-4,6,6a,7,8,9- hexahydroindolo[4,3-fg] quinolin-9-y1) urea Exact Mass: 369.28 | 55% yield LCMS [M]+ 369 |
| Example 3 | 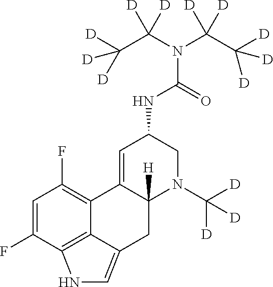 | 3-((6aR,9S)-1,3-difluoro- 7-(methyl-d3)- 4,6,6a,7,8,9- hexahydroindolo[4,3-fg] quinolin-9-y1)-1,1- bis(ethyl-d5) urea Exact Mass: 387.27 | 70% yield LCMS [M]+ 387 |
| Example 4 | 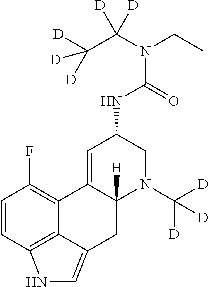 | 1-ethyl-1-(ethyl-d5)-3- ((6aR,9S)-1-fluoro-7- (methyl-d3)-4,6,6a,7,8,9- hexahydroindolo[4,3-fg] quinolin-9-yl) urea Exact Mass: 364.25 | 58% yield LCMS [M]+ 364 |
| Example 5 | 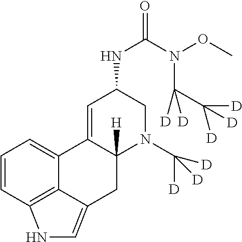 | 1-(ethyl-d5)-1-methoxy-3- ((6aR,9S)-7-(methyl-d3)- 4,6,6a,7,8,9- hexahydroindolo[4,3-fg] quinolin-9-yl) urea Exact Mass: 348.24 | 60% yield LCMS [M]+ 348 |
| Example 6a | 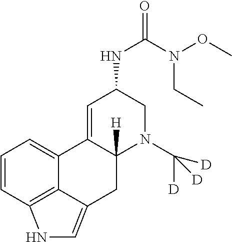 | 1-ethyl-1-methoxy-3- ((6aR,9S)-7-(methyl-d3)- 4,6,6a,7,8,9- hexahydroindolo[4,3-fg] quinolin-9-yl) urea Exact Mass: 343.21 | 77% yield LCMS [M]+ 343 |
| Example 6b | 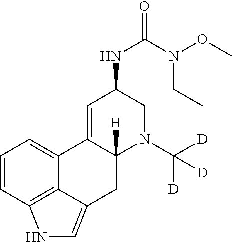 | 1-ethyl-1-methoxy-3- ((6aR,9R)-7-(methyl-d3)- 4,6,6a,7,8,9- hexahydroindolo[4,3-fg] quinolin-9-yl) urea Exact Mass: 343.21 | 39% yield LCMS [M]+ 343 |
| Example 7 | 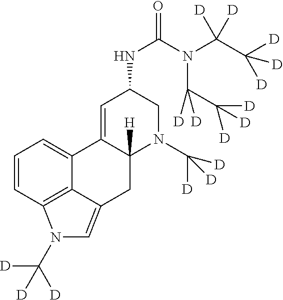 | 3-((6aR,9S)-4,7- bis(methyl-d3)- 4,6,6a,7,8,9- hexahydroindolo[4,3-fg] quinolin-9-yl)-1,1- bis(ethyl-d5) urea Exact Mass: 368.33 | 61% yield LCMS [M]+ 368 |
| Example 8 | 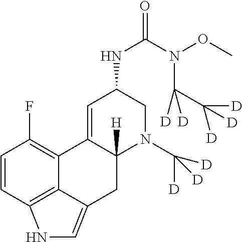 | 1-(ethyl-d5)-3-((6aR,9S)- 1-fluoro-7-(methyl-d3)- 4,6,6a,7,8,9- hexahydroindolo[4,3-fg] quinolin-9-y1)-1- methoxyurea Exact Mass: 366.23 | 84% yield LCMS [M]+ 366 |
| Example 9 | 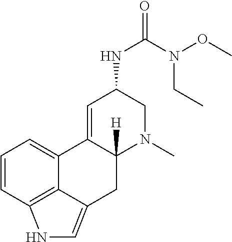 | 1-ethyl-1-methoxy-3- ((6aR,9S)-7-methyl- 4,6,6a, 7,8,9- hexahydroindolo[4,3-fg] quinolin-9-y1) urea Exact Mass: 340.19 | 94% yield LCMS [M]+ 340 |
| Example 10 | 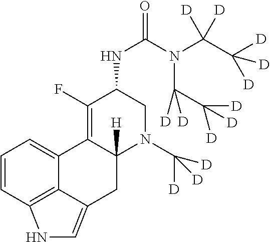 | 1,1-bis(ethyl-d5)-3- ((6aR,9R)-10-fluoro-7- (methyl-d3)-4,6,6a,7,8,9- hexahydroindolo[4,3-fg] quinolin-9-yl) urea Exact Mass: 369.28 | 44% yield LCMS [M]+ 369 |
| Example 11 | 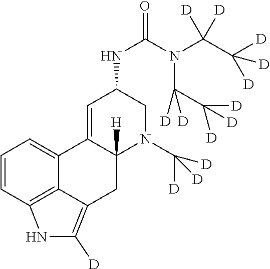 | 1,1-bis(ethyl-d5)-3- ((6aR,9S)-7-(methyl-d3)- 4,6,6a,7,8,9- hexahydroindolo[4,3-fg] quinolin-9-y1-5-d) urea Exact Mass: 352.30 | 6% yield LCMS [M]+ 352 |
| Example 12 | 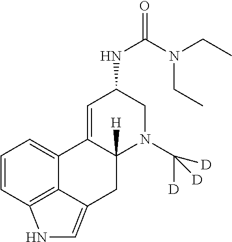 | 1,1-diethyl-3-((6aR,9S)-7- (methyl-d3)-4,6,6a,7,8,9- hexahydroindolo[4,3-fg] quinolin-9-yl) urea Exact mass: 341.23 | 77% yield LCMS [M]+ 342 |

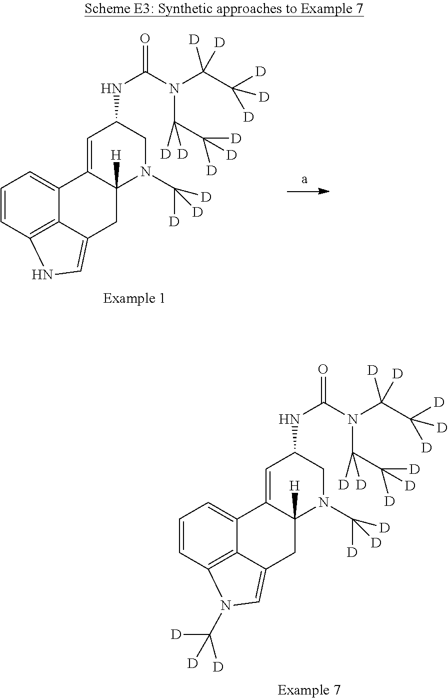

Reagent and conditions: (a) Iodomethane-d3, KOH, Acetone; see patent BE 896122

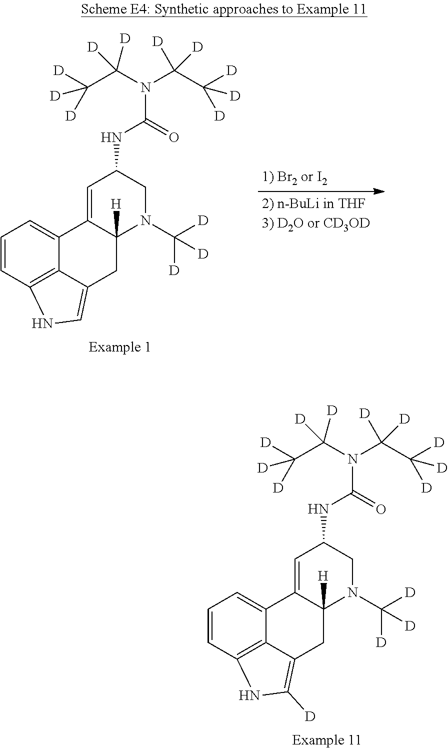

### (i) Alternative Synthetic Route to Example 6 and Example 12

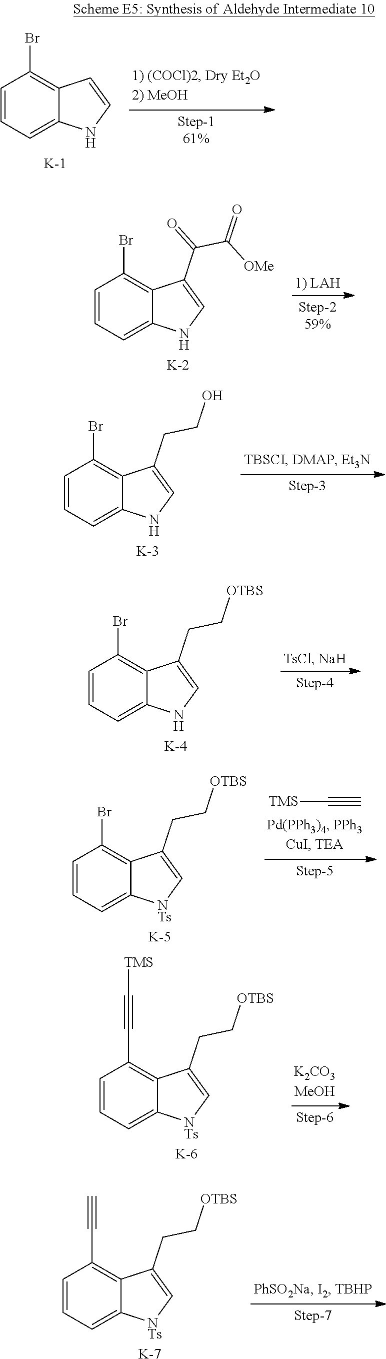

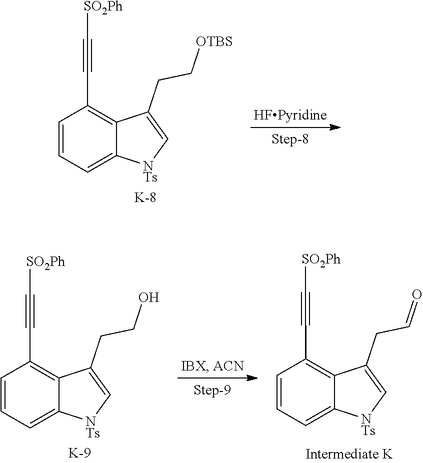

### Step 1: Synthesis of α-Keto methyl ester K-2

To a solution of Compound K-1 (18.7 g, 1.0 eq) in anhydrous diethyl ether (4.5 V) under argon at 0° C. was added Oxalyl chloride (2.0 eq) over 30 min and stirred at RT for 16 h. The mixture was cooled to 0° C. and anhydrous methanol (3.3 eq) carefully added. The suspension was allowed to stir at room temperature for 12 h and filtered. The filtered cake was washed with cold ether and dried to afford 16.4 g of compound K-2; LCMS [M+H]⁺ 282.

### Step 2: Synthesis of Indole alcohol K-3

To a solution of compound K-2 (12 g, 1.0 eq) in THF (5 V) at 0° C. added LAH solution (2.0 M in THF) (3.0 eq) over 30 min and stirred at 60° C. for 4 h. After work-up and purification, 7.3 g of compound K-3 was obtained (85% yield); LCMS [M+H]⁺ 240.

### Step 3: TBS protected alcohol K-4

To a solution of compound K-3 (2.5 g, 1.0 eq) in anhydrous DCM (9 V) under argon at 0° C. was added TEA (1.5 eq), DMAP (0.05 eq), followed by TBSCI (1.05 eq) and stirred at RT for 16 h. After work-up, afford 3.7 g of crude compound K-4 as brown solid. Note: Used for next step without further purification.

### Step 4: N-Tosylated indole K-5

To a solution of crude Compound K-4 (3.6 g, 1.0 eq) in anhydrous THF (15 V) under argon at 0° C. was added 60% NaH (1.1 eq) as a portion-wise and stirred at 0° C. for 15 min and at RT for 1 h. The reaction mixture cooled to 0° C. and TsCl (1.1 eq) added portion-wise, and reaction was stirred at RT for 18 h. After work-up and purification, 4.0 g of compound K-5 was obtained as pale brown color solid.

### Step 5: Alkyne-substituted indole K-6

A solution of compound E5-5 (1.0 g, 1.0 eq) in TEA (10 V) was degassed with argon and added CuI (0.08 eq), PPh₃ (0.08 eq), trimethylsillylacetylene (3.3 eq) & Pd(PPh₃)₄ (0.04 eq) and reaction mixture in sealed tube was stirred at 90-95° C. for 24 h. After work-up and purification, afford 1.1 g of compound K-6. After work-up and purification, 1.1 g of compound K-6 was obtained as a white solid; LCMS [M+H]⁺ 526.

### Step 6: Terminal acetylene K-7

To a solution of compound E5-6 (1.0 g, 1.0 eq) in anhydrous MeOH (10 V) under argon at RT was added K₂CO₃ (0.13 eq) and the mixture was stirred at RT for 18 h. After work-up and purification, 0.52 g of compound K-7 was obtained (71% yield); LCMS [M+H]⁺ 454.

### Step 7: Synthesis of Alkynyl sulfone K-8

To a solution of compound E5-7 (53 g, 1.0 eq) in anhydrous THF (10 V) under argon at 0° C. was added PhSO₂Na (2.0 eq), Iodine (0.5 eq) and THBP (3.0 eq, 70% in Water) stirred at 0° C. for 1 h and at RT for 18 h. After work-up and purification, 42 g of compound K-8 was obtained (81% yield); LCMS [M+H]⁺ 454.

### Step 8: Synthesis of Primary alcohol K-9

To a solution of compound E5-8 (42 g, 1.0 eq) in anhydrous THF (10 V) under argon at 0° C. was added HF-Pyridine (0.2 mL) and stirred at 0° C. for 15 min and at RT for 3 h. After work-up and purification, 25 g of compound K-9 was obtained as a pale yellow solid; LCMS [M+H]⁺ 480.

### Step 9: Synthesis of Aldehyde Intermediate K

To a solution of compound K-9 (34 g, 1.0 eq) in anhydrous ACN (20 V) under argon at RT was added IBX (3.0 eq) and the mixture was stirred at 80° C. for 2 h. Observed 30% SM of SM and 40% of product 10 mass by LCMS. The reaction mixture was cooled to RT and added 2.0 eq of IBX and stirred at 80° C. for 1 h, observed SM consumed by TLC. After work-up and purification, 34 g of Intermediate K was obtained as a brown-yellow solid; LCMS [M+H]⁺ 478.

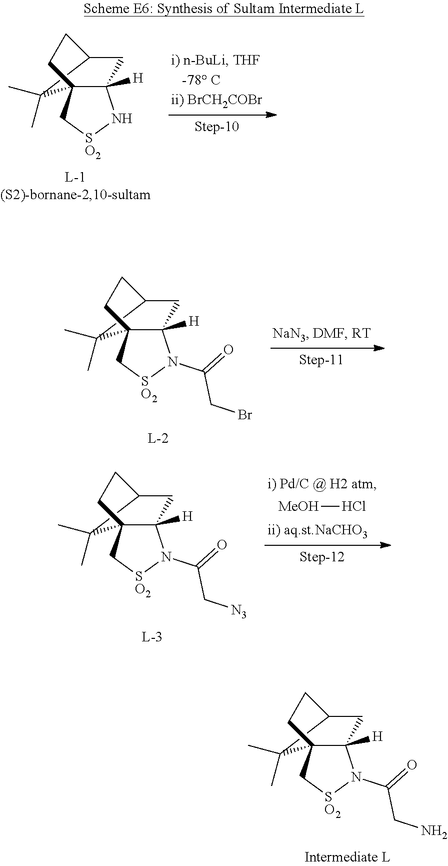

### Step 10: Synthesis of Bromoacetylsultam L-2

To a solution of compound L-1 (70 g, 1.0 eq) in anhydrous THF (27 V) under argon at −78° C. was added N-BuLi (1.6 M in hexane) (1.1 eq) over 30 min and stirred at −78° C. for 1 h. To this mixture was added a solution of bromo acetyl bromide (1.1 eq) in THF (5V) over 1.5 h and the resulting mixture was stirred at −78° C. for another 2 h. After work-up and purification, 95 g of compound L-2 was obtained as an amber solid; LCMS [M+H]⁺ 336.

### Step 11: Synthesis of Azidoacetylsultam L-3

To a solution of compound L-2 (95 g, 1.0 eq) in anhydrous DMF (7.5 V) under argon at RT was added NaN₃ (1.13 eq) and stirred at RT for 16 h. After work-up and purification, 80 g of compound L-3 was obtained as a pale yellow solid; LCMS [M+H]⁺ 299.

### Step 12: Synthesis of Glycylsultam Intermediate L

To a suspension of 10% of Pd/C in Methanol (25 V) and water (5.0 mL) was added compound L-3 (78 g, 1.0 eq) in MeOH (10 V) followed by concentrated HCl (0.75 mL) and the mixture was stirred under hydrogen at RT for 40 h (over weekend). After work-up and purification, 23 g of Intermediate L was obtained as a colorless solid; LCMS [M+H]⁺ 273.

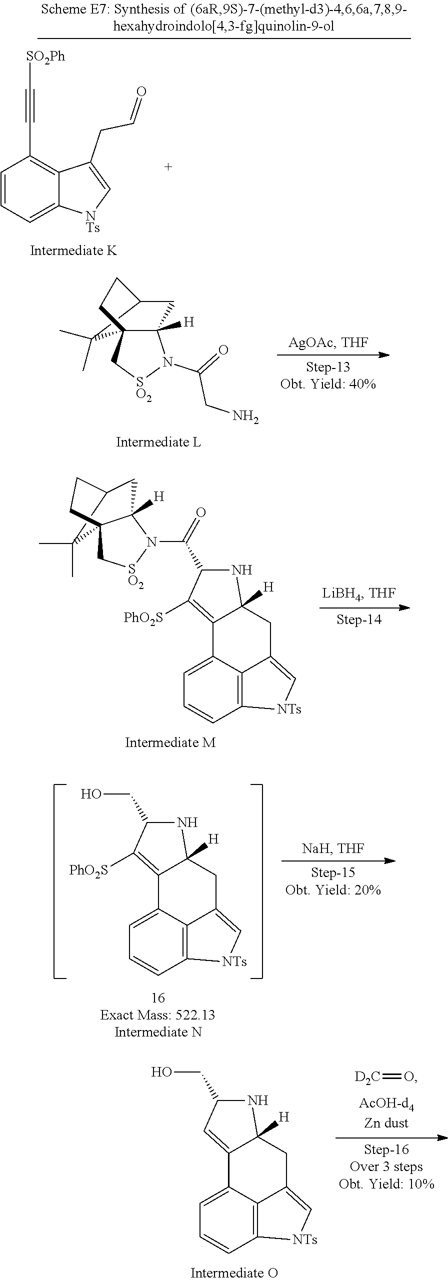

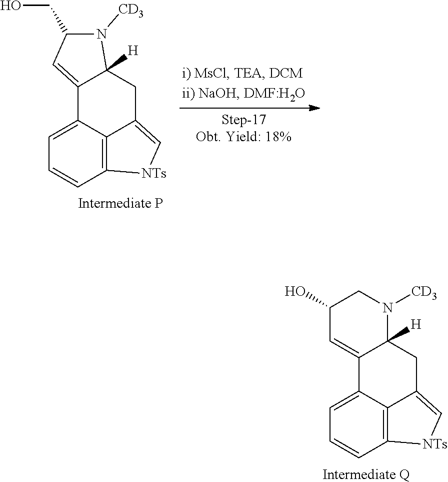

### Step 13: Cyclization to Intermediate M

To a solution of Intermediate K (4.0 g, 1.0 eq) and Intermediate L (1.1 eq) in anhydrous THF (30 V) under argon at RT was added AgOAc (0.1 eq) and stirred in dark at RT for 2 h and monitored by ¹H NMR for the disappearance of the observed aldehyde peak. An additional amount of Intermediate L (0.5 eq) and AgOAc (0.1 eq), was added and the mixture was stirred for another 2 h at RT. After work-up and purification, 2.1 g of Intermediate M was obtained as a brown solid; LCMS [M+H]⁺ 732.

### Step 14/15: Reduction-Elimination to Intermediate O

To a solution of Intermediate M (10 g, 1.0 eq) in anhydrous THF (10 V) at 0° C. under argon was added LiBH₄ (3.0 eq) and stirred at 50° C. for 3 h. An additional 3.0 eq of LiBH₄ and stirred at 50° C. for 16 h. After work-up and purification, the white solid Intermediate N (LCMS [M+H]⁺ 523), was dissolved in dry THF treated with NaH in a single portion under an Ar atmosphere. The resulting mixture was stirred at room temperature until the Intermediate N was completely consumed by LCMS analysis. At this point, the reaction was cooled to 0° C., quenched with 1N HCl and extracted with CH₂Cl₂ (3×50 mL). The combined organic layers were washed with aqueous sat. NaCl solution (50 mL), dried over anhydrous Na₂SO₄ and concentrated under reduced pressure. The crude product was purified by reverse-phase column chromatography to give 1.5 g of Intermediate O, which was obtained as a white solid; LCMS [M+H]⁺ 381.

### Step 16: Introduction of the trideuteromethyl Intermediate P

To a solution of Intermediate 0 (2.4 g, 1.0 eq) in 1,4-dioxane (10 V) at RT under argon was added Acetic acid-d4 (4.0 eq), 20% of D₂CO in D₂O (2.0 eq) and zinc dust (2.0 eq) and stirred at RT for 4 h After work-up and purification, 1.6 g of Intermediate P was obtained as a white solid; LCMS [M+H]⁺ 398.

### Step 17: Synthesis of Alcohol Intermediate Q Through Ring-Expansion

To a solution of Intermediate P (1.6 g, 1.0 eq) in DCM (30 V) at 0° C. added TEA (1.6 eq) and MsCl (1.2 eq) and stirred at 0° C. for 30 min and at RT for 4 h. After work-up, the crude mesylate compound was taken to next step. The mesylate was dissolved in DMF (30 V) and water (2.5 V) and NaOH (5.0 eq) was added. The mixture was then stirred at RT for 4 h. After work-up and purification, 0.55 g of ring-expanded 2° alcohol Intermediate Q was obtained as a yellow-brown solid; LCMS [M+H]⁺ 398.

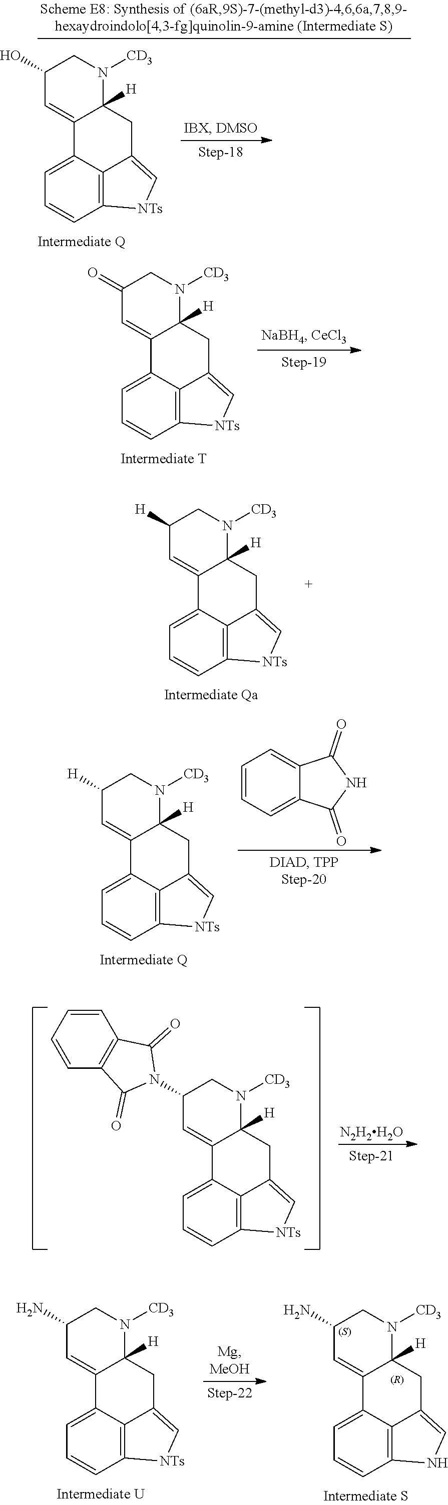

### Step 18: Synthesis of Ketone Intermediate T

To a solution of Intermediate Q (0.55 g, 1.0 eq) in anhydrous DMSO (500 mL) at RT under argon was added IBX (1.2 eq) and stirred at RT for 16 h. After work-up and purification, 0.310 g of ketone Intermediate T was obtained. LCMS [M+H]⁺ 396.

### Step 19: Reduction to Intermediate Qa

To a solution of Intermediate T (0.310 g, 1.0 eq) in MeOH (2.5 mL) at 0° C. under argon was added anhydrous CeCl₃ (2.3 eq) and after 5 min, NaBH₄ (4.0 eq) added and stirred at 10° C. for 1 h. After work-up and purification, 0.2 g of alcohol Intermediate Qa was obtained. LCMS [M+H]⁺ 398.

### Step 20/21: Syntheses of Amine Intermediate U

To a solution of Intermediate Qa (0.2 g, 1.0 eq) in anhydrous THF (0.5 mL) at 0° C. was added Phthalimide (2.0 eq) followed by triphenylphosphine (TPP) (2.0 eq). DIAD (2.0 eq) in THF (50 μL) was then added dropwise and the mixture was stirred for 16 h at RT (NB: Reaction was monitored by LCMS). After work-up, the crude compound was taken directly to the next step. To a solution of crude thalimide intermediate (1.0 eq) was added methanol (0.2 mL) followed by N₂H₂·H₂O (10 eq) and the mixture was stirred for 16 h at RT. After work-up and purification, 0.16 g of amine Intermediate U was obtained. LCMS [M+H]⁺ 397.

### Step 22: Deprotection to the Advanced Intermediate S

To a solution of Intermediate U (160 mg, 1.0 eq) in Dry MeOH (3.0 mL) added activated Mg turnings (20.0 eq) and reaction is heated at 50° C. After work-up and purification, 40 mg of Intermediate S was obtained. LCMS [M+H]⁺ 243.

### (j) Synthesis of 1-ethyl-1-methoxy-3-((6aR,9S)-7-(methyl-d3)-4,6,6a,7,8,9-hexahydroindolo[4,3-fg]quinolin-9-yl)urea (Example 6-R,S-isomer)

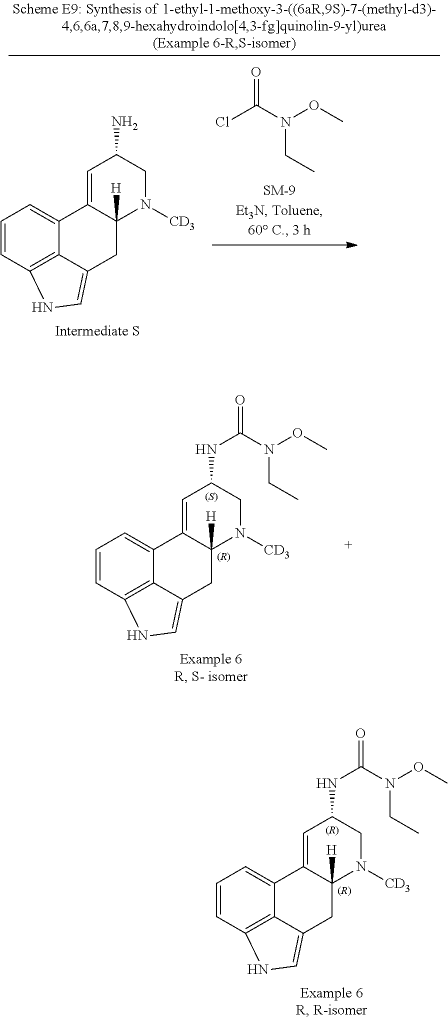

To a solution of Intermediate S (5 mg, 1.0 eq) in anhydrous Toluene (0.8 mL) at RT under argon was added TEA (5.0 eq). After 5 min, the commercially available N-ethyl-N-methoxycarbamoyl chloride (3.0 eq) in dry toluene (0.2 mL) was added and the mixture was stirred at 60° C. for 6 h. After work-up and purification by preparative HPLC, 4.7 mg of the desired compounds (Example 6a-R, S-isomer; LCMS [M+H]⁺ 344) and 2.4 mg of the other diastereoisomer (Example 6b-R, R—Isomer LCMS [M+H]⁺ 344).

### (k) Synthesis of 1,1-diethyl-3-((6aR,9S)-7-(methyl-d3)-4,6,6a,7,8,9-hexahydroindolo[4,3-fg]quinolin-9-yl)urea (Example 12-R,S-isomer)

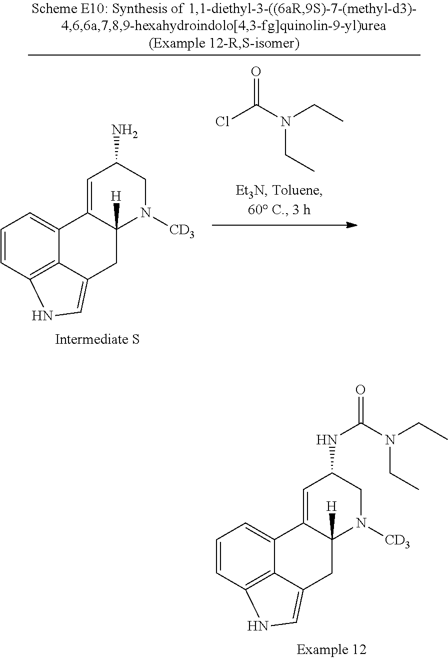

To a solution of Intermediate S (7 mg, 1.0 eq) in anhydrous Toluene (0.5 mL) at RT under argon was added TEA (2.0 eq). After 5 min, the commercially available diethylcarbamoyl chloride (1.5 eq) in dry toluene (0.1 mL) was added and the mixture was stirred at 60° C. for 3 h.

After work-up and purification by preparative HPLC, 1.7 mg of the desired compounds (Example 12-R, S-isomer; LCMS [M+H]⁺ 343)

### II. Biological Evaluation

### (a) 5-HT₂ Receptor Assays

Compounds of the present application bind to the 5-HT₂ receptor subtypes in the following assays: Compounds of the invention are tested on 5-HT_{2A} and 5-HT₂C human recombinant G protein-coupled receptors using a CHO-K1-mt aequorin Gα16 cell line and IP-One assays (Euroscreen Laboratory, Belgium). Dose-response curves for the test compounds are generated over the concentration range of 0.01 to 20,000 nM to determine effective concentration (EC50), inhibitory concentration (IC50) as seen in Table 2, and relative degree of agonistic and antagonistic response (“relative response”). Compound binding was calculated as a % inhibition of the binding of a radioactively labeled ligand specific for each receptor. Results with inhibition >50% were considered to represent significant effects. In each experiment, the respective reference compound was tested in parallel with the test compounds, and the data were compared with previous values determined at Eurofins.

TABLE 2
Representative examples of the compounds of the invention
showing their 5-HT2A and 5-5-HT2C binding profiles

| Compound ID | 5-HT2C IC₅₀ (nM) | 5-HT2A IC₅₀ (nM) |
|---|---|---|
| Lisuride | 26 | 0.97 |
| Example 6a | 96 | 2.6 |
| Example 6b | 312 | 6.5 |
| Example 12 | 76 | 0.04 |
| Serotonin (control) | 278 | — |
| Ketanserin (control) | — | 2 |

### Procedure for the 5-HT_{2A} Binding Assay

### Materials

Ketanserin hydrochloride, [Ethylene-3H]—was purchased from PerkinElmer. Ketanserin was purchased from MedChemExpress. Bovine Serum Albumin (BSA), calcium chloride (CaCl₂)), and polyethylenimine, branched (PEI) were purchased from Sigma. Tris(hydroxymethl)aminomethane (Tris) was purchased from Alfa Aesar.

### Instruments and Consumables

Microbeta² microplate counter, MicroBeta Filtermate-96, and UniFilter-96 GF/C were purchased from PerkinElmer. TopSeal was purchased from Biotss. Seven Compact pH meter was purchased from Mettler Toledo. Ultrapure water meter was purchased from Sichuan Ulupure. Benchtop Centrifuge was purchased from Hunan Xiangyi. Microplate shaker was purchased from Allsheng. 384-Well Polypropylene Microplate was purchased from Labcyte. 96 round well plate was purchased from Corning. 96 round deep well plate was purchased from Axygen. Echo was purchased from LABCYTE.
1. Prepare the assay buffer following the table below:

| Reagent | Concentration |
|---|---|
| Tris | 50 mM |
| CaCl₂ | 4 mM |
| BSA | 0.1% (w/v) |

Adjust pH to 7.4 followed by 0.2 μM sterile filtration

2. Preparation of 8 doses of reference starting from 0.3 mM stock solution and test compounds starting from 10 mM stock solution and dilutions with 100% (v/v) DMSO.
3. Pretreatment of UniFilter-96 GF/B plate
   a. Add 50 μL/well of 0.5% (v/v) PEI to UniFilter-96 GF-B plates. Seal the plates and incubate at 4° C. for 3 hrs.
   b. After incubation, wash the plates 2 times with ice-cold wash buffer (50 mM Tris, pH 7.4).
4. Preparation of assay plates
   a. Dilute cell membrane with assay buffer and add 330 μL/well to 96 round deep well plates to reach a concentration of 40 μg/well.
   b. Prepare 8 concentrations of reference or test compounds with assay buffer and add 110 μL/well to 96 round deep well plates.
   c. Dilute [3H]-ketanserin with assay buffer to 5 nM (5× final concentration) and add 110 μL/well to 96 round deep well plates.
5. Centrifuge the plate at 1000 rpm for 30 secs and then agitate at 600 rpm, R.T. for 5 min.
6. Seal the plate and incubate the plate at 27° C. for 90 min.
7. Stop the incubation by vacuum filtration onto GF/C filter plates followed by 4 times washing with ice-cold wash buffer (50 mM Tris, pH 7.4).
8. Dry the plates at 37° C. for 45 min.
9. Seal the filter plates and add 40 μL/well of scintillation cocktail.
10. Read the plate by using a Microbeta² microplate counter.

### Data Analysis

11. For reference and test compounds, the results are expressed at % inhibition, using the normalization equation: N=100−100*(U−C₂)/(C₁−C2), where unknown value, C₁, is the average of high controls, and C₂ is the average of low controls. The IC₅₀ is determined by fitting percentage of inhibition function of compound concentrations with Hill equation using XLfit.

### Procedure for the 5-HT₂C Binding Assay

### Materials

[3H]-Mesulergine was purchased from PerkinElmer. Serotonin HCl was purchased from Selleck. Calcium chloride (CaCl₂)) and polythyleneimine (PEI) were purchased from Sigma. Tris(hydroxymethyl)aminomethane (Tris) was purchased from Alfa Aesar).

### Instruments and Consumables

Microbeta² microplate counter, MicroBeta Filtermate-96, and UniFilter-96 GF/C were purchased from PerkinElmer. TopSeal was purchased from Biotss. Seven Compact pH meter was purchased from Mettler Toledo. Ultrapure water meter was purchased from Sichuan Ulupure. Benchtop Centrifuge was purchased from Hunan Xiangyi. Microplate shaker was purchased from Allsheng. 384-Well Polypropylene Microplate was purchased from Labcyte. 96 round well plate was purchased from Corning. Echo was purchased from LABCYTE.
1. Prepare the assay buffer following the table below:

| Reagent | Concentration |
|---|---|
| Tris | 50 mM |
| CaC1₂ | 4 mM |

Adjust pH to 7.4 followed by 0.2 μM sterile filtration

2. Preparation of 8 doses of reference starting from 100 mM stock solution and test compounds starting from 10 mM stock solution as requested by dilutions with 100% (v/v) DMSO.
3. Pretreatment of UniFilter-96 GF/C plate.
   a. Add 50 μL/well of 0.5% (v/v) PEI to UniFilter-96 GF/C plates. Seal the plates and incubate at 4° C. for 3 hrs.
   b. After incubation, wash the plates 2 times with ice-cold wash buffer (50 mM Tris, pH 7.4).
4. Preparation of assay plates
   a. Prepare 8 concentrations of reference or teset compounds and add 50 μL/well to 96 round deep well plates.
   b. Dilute cell membrane with assay buffer and add 100 μL/well to 96 well plates to reach a concentration of 0.5 unit/well.
   c. Dilute [3H]-Mesulergine with assay buffer to 6 nM (4× final concentration) and add 50 μL/well to 96 round well plates.
5. Centrifuge the plate at 1000 rpm for 30 ecs and then agitate at 600 rpm, R.T. for 5 min.
6. Seal the plates and incubate the plate at 27° C. for 60 min.
7. Stop the incubation by vacuum filtration onto GF/C filter plates followed by 6 times washing with ice-cold wash buffer (50 mM Tris, pH 7.4).
8. Dry the plates at 37° C. for 45 min.
9. Seal the filter plates and add 40 μL/well of scintillation cocktail.
10. Read the plate by using a Microbeta² microplate counter

### Data Analysis

11. For reference and test compounds, the result are expressed as % inhibition, using the normalization equation: N=100-100*(U−C₂)/(C₁-C₂), where U is the unknown value, C₁ is the average of high controls, and C₂ is the average of low controls. The IC₅₀ is determined by fitting percentage of inhibition as a function of compound concentrations with Hill equation using XLfit.
(b) Microsomal stability Assays

### Liver Microsomal Metabolic Stability

In Phase I analysis, test compounds are incubated at a final concentration of 1 μM (this concentration is assumed to be well below the Kₘ values to ensure linear reaction conditions i.e. to avoid saturation). Working stocks are initially diluted to a concentration of 40.0 μM in 0.1 M potassium phosphate buffer (pH 7.4) before addition to the reaction vials. CD-1 mouse (male) or pooled human liver microsomes (Corning Gentest) are utilized at a final concentration of 0.5 mg/mL (protein). Duplicate wells are used for each time point (0 and 60 minutes). Reactions are carried out at 37° C. in an orbital shaker at 175 rpm, and the final DMSO concentration is kept constant at 0.10%. The final volume for each reaction is 100 μL, which includes the addition of an NADPH-Regeneration Solution (NRS) mix. This NRS mix is comprised of glucose 6-phosphate dehydrogenase, NADP+, MgCl₂, and glucose 6-phosphate. Upon completion of the 60 minute time point, reactions are terminated by the addition of 2-volumes (200 μL) of ice-cold, acetonitrile containing 0.5% formic acid and internal standard. Samples are then centrifuged at 4,000 rpm for 10 minutes to remove debris and precipitated protein. Approximately 150 μL of supernatant is subsequently transferred to a new 96 well microplate for LC/MS analysis:

Narrow-window mass extraction LC-MS analysis is performed for all samples in this study using a Waters Xevo quadrupole time-of-flight (QTof) mass spectrometer to determine relative peak areas of test compounds. The percent remaining values are calculated using the following equations:

$$\% remaining = ( A ) / {A}_{0} \times 100$$

where A is area response after incubation and A₀ is area response at initial time point

For intrinsic clearance assay, incubation mixtures contain probe substrate, liver microsomes and an NADPH regenerating system (1.3 mM NADP+, 3.3 mM glucose 6-phosphate, 0.4 U ml-1 glucose 6-phosphate dehydrogenase, 3.3 mM magnesium chloride) in 0.1 M potassium phosphate buffer (pH 7.4). CD-1 mouse (male) or pooled human liver microsomes (Corning Gentest) are utilized at a final concentration of 0.5 mg/mL (protein). 12.5 μL of each drug solution are placed into a well of 96 well plate. Reactions are initiated by the addition of activated microsome solutions (500 μL) to drug solutions. Reactions are carried out at 37° C. in an orbital shaker at 175 rpm, and the final DMSO concentration is kept constant at 0.1%. Test compounds are incubated at a final concentration of 1 μM. 50 μL of aliquots of reaction mixtures are quenched by mixing with two parts of stop solution (internal standard containing 0.5% formic acid in acetonitrile) at appropriate time-points and mixed well. Then, solutions are centrifuged at 4000 rpm for 10 min. Supernatants are transferred to a new 96-well plate and analyzed by a Waters Q-TOF mass spectrometer coupled with an UPLC System. Recovery analysis is performed using relative peak areas and narrow window mass extraction. The ln(% remaining) is plotted against time and the gradient of the line determined.

Elimination Constant (k)=−slope

Half-life (t1/2)(min)=ln2/k=0.693/k

V(μL/mg)=volume of incubation (μL)/protein in the incubation (mg)

Intrinsic Clearance (CLint)(μL/min/mg protein)=V·0.693/t½=V·k

### III. Preparation of Pharmaceutical Dosage Forms

### (a) Oral Capsule

The active ingredient is a compound of Table 1, or a pharmaceutically acceptable salt or solvate thereof. A capsule for oral administration is prepared by mixing 1-1000 mg of active ingredient with starch or other suitable powder blend. The mixture is incorporated into an oral dosage unit such as a hard gelatin capsule, which is suitable for oral administration.

### (b) Solution for Injection

The active ingredient is a compound of Table 1, or a pharmaceutically acceptable salt thereof, and is formulated as a solution in sesame oil at a concentration of 50 mg-eq/mL.

The examples and embodiments described herein are for illustrative purposes only and various modifications or changes suggested to persons skilled in the art are to be included within the spirit and purview of this application and scope of the appended claims.

1-34. (canceled)

35. A compound, or pharmaceutically acceptable salt or solvate thereof, having the structure of Formula (II): 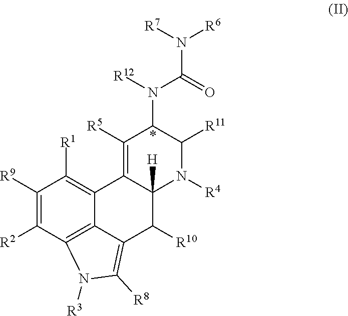
wherein,
R¹ is H, halogen, alkoxy, haloalkoxy, or haloalkyl;
R² is H, halogen, alkoxy, haloalkoxy, or haloalkyl;
R³ is H, alkyl, or deteuroalkyl;
R⁴ is alkyl or deuteroalkyl;
R⁵ is H or halogen;
R⁶ is optionally substituted C₁₋₆ alkyl, optionally substituted C₁₋₆ deuteroalkyl, or optionally substituted C₁-6 alkoxy;
R⁷ is optionally substituted C₁₋₆ alkyl, optionally substituted C₁-6 deuteroalkyl, or optionally substituted C₁-6 alkoxy;
R⁸ is H or D;
R⁹ is H, halogen, alkoxy, haloalkoxy, or haloalkyl;
R¹⁰ is H, D, alkyl, cycloalkyl, or deuteroalkyl;
R¹¹ is H, D, alkyl, cycloalkyl, or deuteroalkyl;
R¹² is H, alkyl, cycloalkyl, or deuteroalkyl;
* indicates R or S stereochemistry;
provided that when each of R¹, R², R³, R⁵, and R⁸ are H and R⁴ is CH₃, then R⁶ and R⁷ are not both CH₂CH₃.

36. The compound, or pharmaceutically acceptable salt or solvate thereof, of claim 35, wherein R⁹, R¹⁰, R¹¹, and R¹² are H.

37. The compound, or pharmaceutically acceptable salt or solvate thereof, of claim 36, having the structure of Formula (I):

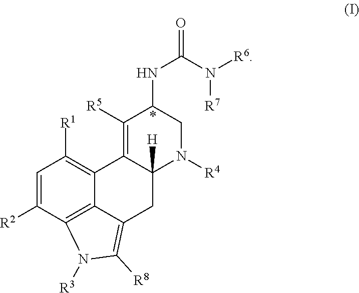

38. The compound, or pharmaceutically acceptable salt or solvate thereof, of claim 35, wherein * indicates S stereochemistry.

39. The compound, or pharmaceutically acceptable salt or solvate thereof, of claim 36, having the structure of Formula (Ia): 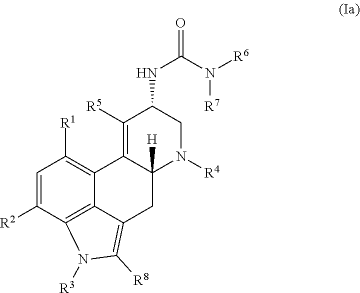
wherein,
R¹ is H, halogen, OMe, CF₃, OCHF₂, or OCF₃;
R² is H, halogen, OMe, CF₃, OCHF₂, or OCF₃;
R³ is H, CH₃ or CD₃;
R⁴ is CH₃ or CD₃; and
R⁵ is H or F.

40. The compound, or pharmaceutically acceptable salt or solvate thereof, of claim 35 wherein R¹ is H or F.

41. The compound, or pharmaceutically acceptable salt or solvate thereof, of claim 35 wherein R² is H or F.

42. The compound, or pharmaceutically acceptable salt or solvate thereof, of claim 35 wherein R³ is H, CH₃ or CD₃.

43. The compound, or pharmaceutically acceptable salt or solvate thereof, of claim 35 wherein R⁴ is CH₃ or CD₃.

44. The compound, or pharmaceutically acceptable salt or solvate thereof, of claim 35 wherein R⁵ is F or H.

45. The compound, or pharmaceutically acceptable salt or solvate thereof, of claim 35 wherein R⁶ is optionally substituted C₁-6 alkyl or optionally substituted C₁-6 alkoxy.

46. The compound, or pharmaceutically acceptable salt or solvate thereof, of claim 35 wherein R⁷ is optionally substituted C₁-6 alkyl or optionally substituted C₁-6 alkoxy.

47. The compound, or pharmaceutically acceptable salt or solvate thereof, of claim 35, wherein R⁶ is C₁-6 deuteroalkyl.

48. The compound, or pharmaceutically acceptable salt or solvate thereof, of claim 35, wherein R⁷ is C₁-6 deuteroalkyl.

49. The compound, or pharmaceutically acceptable salt or solvate thereof, of claim 35 wherein R⁸ is H.

50. The compound, or pharmaceutically acceptable salt or solvate thereof, of claim 35 wherein R¹, R², R³, R⁵, and R⁸ are H.

51. The compound, or pharmaceutically acceptable salt or solvate thereof, of claim 35 having the structure of:

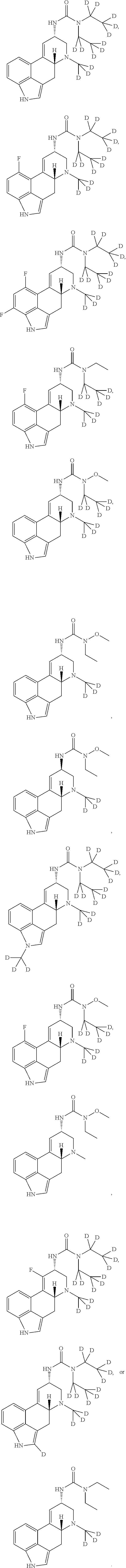

52. A pharmaceutical composition comprising a compound, or pharmaceutically acceptable salt or solvate thereof according to claim 35 and a pharmaceutically acceptable excipient.

53. The pharmaceutical composition of claim 52, wherein in the compound, or pharmaceutically acceptable salt or solvate thereof, * indicates S stereochemistry.

54. The pharmaceutical composition according to claim 52, wherein the compound, or pharmaceutically acceptable salt or solvate thereof, has the structure of Formula (Ia): 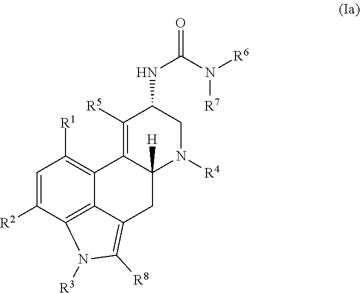
wherein,
R¹ is H, halogen, OMe, CF₃, OCHF₂, or OCF₃;
R² is H, halogen, OMe, CF₃, OCHF₂, or OCF₃;
R³ is H, CH₃ or CD₃;
R⁴ is CH₃ or CD₃; and
R⁵ is H or F.
